(19) United States
(12) Patent Application Publication
(10) Pub. No.: US 2016/0058725 A1
(43) Pub. Date: Mar. 3, 2016

(54) Topical Formulations and Methods for Drug Delivery

(71) Applicant: BIOCHEMICS, INC., Danvers, MA (US)

(72) Inventors: Stephen G. Carter, Andover, MA (US); Zhen Zhu, Andover, MA (US); Gyula Varadi, Watertown, MA (US)

(21) Appl. No.: 14/774,466

(22) PCT Filed: Mar. 14, 2014
(86) PCT No.: PCT/US2014/029240

§ 371 Date: Sep. 10, 2015

Related U.S. Application Data
(60) Provisional application No. 61/790,126, filed on Mar. 15, 2013.

(51) Int. Cl. A61K 31/198; A61K 31/455; A61K 31/195; A61K 9/00; A61K 31/4174; A61K 31/192; A61K 31/047; A61K 31/675; A61K 31/05

(52) U.S. Cl. CPC A61K 31/198; A61K 31/675; A61K 31/455; A61K 31/195; A61K 31/05; A61K 31/4174; A61K 31/192; A61K 31/047; A61K 9/0004; A61K 9/0014

(57) ABSTRACT

Disclosed are topical formulations for delivery of an active ingredient to a patient. The formulation comprises components including: an active ingredient; a vasoactive agent; and a chelator, wherein the components are selected so that none of the other components is sequestered by the chelator. In some embodiments, the formulation comprises an osmolarity that is greater than about 345 milliOsmoles/liter (345 mOsM). In some embodiments, the formulation further comprises an osmolyte, wherein the osmoyte does not include an ion with a valency higher than monovalency. In some embodiments, the osmolyte in the formulation comprises an osmolarity that is greater than about 290 milliOsmoles/liter (290 mOsM). Also disclosed are methods for using the formulations, and components thereof, kits comprising the components of the formulation, and methods for manufacturing a medicament comprising components of the formulations.

### CROSS-REFERENCE TO RELATED APPLICATION

This application claims benefit of United States Provisional Patent Application Serial No. 61/790,126, filed on Mar. 15, 2013, the entire contents of which application is hereby incorporated by reference.

### TECHNICAL FIELD

The present invention relates to drug delivery formulations and methods, and more particularly to topical drug delivery formulations and methods for delivery or treatments through or into the skin tissue and into other tissues.

### BACKGROUND

The topical delivery of active ingredients through the skin or other body surfaces is attractive to patients for a variety of reasons. In addition to convenience, topical formulations avoid the irritation of gastrointestinal tract that often accompany pills and capsules. Furthermore, for topical delivery to the skin, by not breaking the patient's skin (e.g., with a hypodermic needle), a topical formulation can avoid patient discomfort (and patient deterrence) and the possibility of infection.

However, for a topical drug delivery formulation to be effective, it has to be able to traverse the layer of cells at the surface of the tissue or organ. Where the topical drug is applied to the skin, the delivery formulation must break through the body's strongest physical barrier against the outside world: the skin. Skin has two major functions. First, skin protects the body from external insults (e.g. harmful substances and microorganisms). And second, skin contains all body fluids. Consequently, skin is extremely strong and yet flexible.

Skin is comprised of multiple layers, with each layer having its own sub-layers (see FIG. 1). The two outermost layers of skin are the epidermis (outermost) and the dermis. Cells of the epidermis and dermis, whether living or dead, are called “skin cells”. For transdermal delivery, an active ingredient must pass through the epidermis to reach the microcirculation (e.g., via capillary blood vessels) of the dermis.

Cells of the epidermis are epithelial cells. As shown in FIGS. 1 and 2, the epidermis itself has multiple sub-layers (see FIG. 2), the outermost being the stratum corneum which overlies the stratum lucidum (in palm and soles) which (if present) overlies the stratum granulosum, which overlies the stratum spinosum, which overlies the stratum basale layer. The epidermis is separated from the dermis by a basement membrane.

The outermost layer, the stratum corneum, is made of mostly dead cells (corneocytes) that lack nuclei and organelles. Corneocytes of the stratum corneum contain a dense network of keratin, a protein that helps keep the skin hydrated by preventing water evaporation and alterations in the osmolarity of the underlying bodily fluids. Corneocytes can also absorb water, further aiding in hydration. The thickness of the stratum corneum varies throughout the body. In the palms of the hands and the soles of the feet this layer is typically thicker. In general, the stratum corneum contains 15 to 20 layers of dead cells (i.e., 15-20 layers of corneocytes). The stratum corneum typically has a thickness of between about 10 and 40 μm.

The stratum corneum is formed as proliferating keratinocytes (which form in the stratum basale) migrate upward (or outward) through the epidermis toward the surface, finally reaching the stratum corneum after approximately 14 days. During cornification (i.e., the process where living keratinocytes are transformed into non-living corneocytes), the cell membrane is replaced by a layer of ceramides which become covalently linked to an envelope of structural proteins called the cornified envelope. This envelope complex surrounds cells in the stratum corneum and contributes to the skin's barrier function. Corneodesmosomes (modified desmosomes) facilitate cellular adhesion by linking adjacent cells. These complexes are degraded by proteases, eventually permitting cells to be shed at the surface. Desquamation and formation of the cornified envelope are both required for the maintenance of skin homeostasis. A failure to correctly regulate these processes leads to the development of skin disorders.

A successful topical delivery system must be able to transmit the active ingredient through the cells at the surface of the organ and into the underlying tissue.

### SUMMARY OF THE EMBODIMENTS

The present invention provides methods and reagents (including compositions) that allow topical delivery of an active ingredient to a patient in need thereof.

Accordingly, In a first aspect, the invention provides a topical formulation for delivery of an active ingredient to a patient, comprising components including: an active ingredient; a vasoactive agent; and a chelator, wherein the components are selected so that none of the other components is sequestered by the chelator, wherein the formulation comprises an osmolarity that is greater than about 345 milliOsmoles/liter (345 mOsM). In some embodiments, the formulation further comprises an osmolyte, wherein the osmolyte does not include an ion with a valency higher than monovalency. In some embodiments, the osmolyte is a sugar osmolyte.

In another aspect, the invention provides a topical formulation for delivery of an active ingredient to a patient, comprising components including: an active ingredient; a vasoactive agent; a chelator, wherein the components are selected so that none of the other components is sequestered by the chelator; and an osmolyte, wherein the osmolyte does not include an ion with a valency higher than monovalency, wherein the osmolyte comprises an osmolarity that is greater than about 290 milliOsmoles/liter (290 mOsM).

In some embodiments, the active ingredient, vasoactive agent, chelator (and optional osmolyte) are mutually exclusive. In some embodiments, the formulation further comprises a lipid or a penetration enhancer. In some embodiments, the chelator is EDTA or EGTA. In some embodiments, the concentration of the chelator is between about 0.05% w/w and about 10% w/w, or is between about 1% w/w and about 5% w/w.

In some embodiments, the vasoactive agent is a vasodilator. In some embodiments, the topical application of the formulation to the patient does not permanently damage cells of the patient.

In a further aspect, the invention provides a method for topical delivery of an active ingredient to a patient in need thereof, comprising applying an effective amount of the formulation described herein to a topical application site of the patient.

In another aspect, the invention provides a method for topical delivery of an active ingredient to a patient in need thereof, comprising: applying an effective amount of the active ingredient to a topical application site of the patient; applying a first amount of a vasoactive agent to the topical application site; and applying a second amount of a chelator to the topical application site, wherein the vasoactive agent and the active ingredient are selected so none of the vasoactive agent and the active ingredient is sequestered by the chelator. In some embodiments, the first amount and second amount, together with the effective amount, result in an osmolarity of the components at the topical application site of at least 345 mOsmol/liter. In some embodiments, the method further comprises applying a third amount of an osmolyte to the topical application site, wherein the osmolyte does not include an ion with a valency higher than monovalency. In some embodiments, the osmolyte is present at an osmolarity of at least 290 mOsmol/liter. In some embodiments, the patient is human.

In some embodiments, the osmolyte is a sugar osmolyte. In some embodiments, the active ingredient, vasoactive agent, and chelator are applied sequentially. In some embodiments, the active ingredient, vasoactive agent, and chelator are applied together.

In some embodiments, the cells at the topical application site are not permanently damaged. In some embodiments, the topical application site is on a skin surface of the patient. In some embodiments, the topical application site is on a tissue surface of a patient. For example, the tissue surface may be the surface of a solid tumor or may be the surface of an organ.

In another aspect, the invention provides a kit for topical delivery of an active ingredient to a patient is provided. The kit's components include a vasoactive agent, a chelator, an active ingredient; and optionally one or more additional components (e.g., a transpiration barrier and/or an osmolyte), where the components are selected so that none of the other components is sequestered by the chelator and a set of written instructions for use, by or on said patient, of the components of the kit according to one of the methods of topical delivery described herein. In some embodiments, the osmolarity of all the components of the kit is greater than about 345 milliOsmol/liter. In some embodiments, the osmolarity of the osmolyte in the kit is greater than about 290 milliOsmol/liter.

In another aspect, the invention provides a method of manufacturing a medicament for topical delivery of an active ingredient. The method includes combining components including a vasoactive agent, a chelator, an active ingredient, and optionally one or more additional components (e.g., a transpiration barrier or an osmolyte), where the components are selected so that none of the other components is sequestered by the chelator. In some embodiments, all of the components are present in sufficient amounts to raise the osmolarity of the medicament containing the active ingredient to at least about 345 mOsmol/liter. In some embodiments, the osmolyte is present in the medicament at an osmolarity of at least about 290 milliOsmol/liter.

### BRIEF DESCRIPTION OF THE DRAWINGS

The foregoing features of embodiments will be more readily understood by reference to the following detailed description, taken with reference to the accompanying drawings, in which:

FIG. 1 is a schematic diagram showing a cross section view of the skin of a mammal (in this case, a human). Skin is comprised of an upper epidermis layer overlaying a dermis layer comprising connective tissue and blood vessels. The dermis layer, in turn, overlays the subcutaneous tissue comprising adipose tissue.

FIG. 2 is a schematic diagram showing a cross section view of the epidermis of a mammal (in this case, a human). The sublayers of epidermis (from outermost inward) are the stratum corneum, the stratum lucidum the stratum granulosum, the stratum spinosum, and the stratum basale.

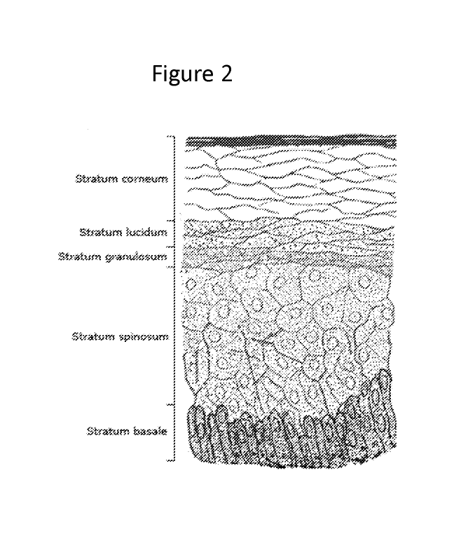

FIG. 3A is a flow diagram showing a method for transdermal drug delivery of a formulation comprising a chelator, a vasoactive agent, and active ingredient (where the combined osmolarity of the formulation is greater than 345 mOsM) in accordance with a non-limiting embodiment of the invention.

FIG. 3B is a flow diagram showing a method for transdermal drug delivery of a formulation comprising a chelator, a vasoactive agent, an active ingredient, and an osmolyte (whose osmolarity is greater than 290 mOsM) in accordance with a non-limiting embodiment of the invention.

FIG. 4A is a schematic showing the effect of a non-limiting formulation comprising a chelator, a vasoactive agent, and an active ingredient (where the combined osmolarity of the formulation is greater than 345 mOsM) on the tight junction formed by two adjacent skin cells.

FIG. 4B is a schematic showing the effect of a non-limiting formulation comprising a chelator, a vasoactive agent, an active ingredient, and an osmolyte (whose osmolarity is greater than 290 mOsM) on the tight junction formed by two adjacent skin cells.

FIGS. 5A-5C are electron microscopy images of guinea pig skin taken from the back of the animal after no treatment (FIG. 5A), 30 minutes after a single (one time) topical application of a formulation comprising a component with a chelating activity (FIG. 5B), and 60 minutes (FIG. 5C) after a single (one time) topical application of a formulation as described below in Example 4 comprising a component with a divalent cation chelating activity.

FIG. 6 is a bar graph showing the concentration of a non-limiting active ingredient, ibuprofen, in blood plasma (in ug/ml) following topical application of Formulation A (containing ibuprofen plus a vasodilator), Formulation B (containing ibuprofen plus an osmolyte), and Formulation C (containing ibuprofen, a vasodilator, and an osmolyte).

### DETAILED DESCRIPTION OF SPECIFIC EMODIMENTS

In some embodiments, the present disclosure is based on the discovery that the inclusion of a chelator and a vasoactive agent in a topical formulation that is hypertonic to the patient will facilitate the delivery of an active ingredient in the topical formulation to that patient

Embodiments described herein can be useful for medical conditions such as but not limited to basal cell carcinomas, melanoma, cervical carcinomas, cervical condylomas, genital warts, herpetic lesions, diabetic neuropathy, chemotherapy-derived neuropathy, general neuropathy, benign prostatic hypertrophy, solid tumors, psoriasis, and eczema. In some embodiments, the active ingredient is a sirtuin inhibitor or sirtuin activator and the formulation is applied to the skin of a patient to treat one of these medical conditions. In some embodiments, the formulation can be applied to a region of the skin or tissue associated with the medical condition. In some embodiments, the formulation is cosmetically suitable in that it can be applied to the skin without detrimentally affecting the appearance of the skin. For example, the formulation may include pigment or dye to match the skin tone of the patient.

The published patents, patent applications, websites, company names, and scientific literature referred to herein establish the knowledge that is available to those with skill in the art and are hereby incorporated by reference in their entirety to the same extent as if each was specifically and individually indicated to be incorporated by reference. Any conflict between any reference cited herein and the specific teachings of this specification shall be resolved in favor of the latter.

The further aspects, advantages, and embodiments of the invention are described in more detail below. The definitions used in this specification and the accompanying claims shall have the meanings indicated, unless the context clearly otherwise requires. Any conflict between an art-understood definition of a word or phrase and a definition of the word or phrase as specifically taught in this specification shall be resolved in favor of the latter. As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Technical and scientific terms used herein have the meaning commonly understood by one of skill in the art to which the present disclosure pertains, unless otherwise defined. Reference is made herein to various methodologies and materials known to those of skill in the art. Standard reference works setting forth the general principles of recombinant DNA technology, all of which are incorporated herein by reference in their entirety, include Rose and Post, Clinical Physiology of Acid-Base and Electrolyte Disorders (5^{th} Ed.), McGraw-Hill, 2001, Ansel's Pharmaceutical Dosage Forms and Drug Delivery Systems (9^{th} Ed.), eds. L. V. Allen, Jr., N. G. Popovich, and H. C. Ansel, Lippincott Williams & Wikins, 2011, J. P. Remington, Remington: The Science and Practice of Pharmacy (21st Ed.), ed. Randy Hendrickson, Lippincott Williams & Wilkins, 2005, and Transdermal and Topical Drug Delivery Systems, eds. T. Ghosh, W. Pfister, and S. I. Yum., CRC Press, 1997.

As discussed above, skin is one of the strongest barriers in a multicellular organism. Skin protects the organism from the external world (e.g., resisting infection) and retains water in the organism. Skin is made of multiple layers of skin cells, both living and dead, and the skin cells in each layer are tightly held together. The space between any two cells is called the interstitial space. In skin, the interstitial spaces are held together very tightly by the interactions of cell surface receptors on adjacent skin cells.

Once in the dermis, the active ingredient can either enter the systemic circulatory system of the patient by, for example, entering the blood vessels (capillaries and veins) in the dermis or via the lymphatic system, or can remain locally in the area in which the formulation containing the active ingredient was applied.

For topical application of an active ingredient (e.g., a local antibiotic) or a formulation containing the active ingredient on the skin, in order to get into the dermis without permanently damaging any skin cells, the active ingredient must be carried past the cells in the epidermis. In accordance with the formulations and methods described herein, this can be accomplished by either incorporating the active ingredient in a formulation that allows the components in the formulation (including the active ingredient) to move within the interstitial spaces past the skin cells in the epidermis and the basement membrane, so as to enter the dermis, or by applying the active ingredient either simultaneously with or sequentially with other components whereby the combination of these components with the active ingredients creates a hypertonic condition that allows the components and active ingredients to move within the interstitial spaces past the skin cells in the epidermis and the basement membrane, so as to enter the dermis.

Similarly, for topical application of an active ingredient (e.g., an anti-cancer drug) or a formulation containing the active ingredient on the surface of an organ or tissue (e.g., the surface of a solid tumor), in order to get into the core of the tumor without permanently damaging any cells at the tumor surface.

Accordingly, in a first aspect, provided is a topical formulation for transdermal delivery of an active ingredient to a patient, comprising components that include an active ingredient activity; a vasoactive agent; and a chelator, where the components are selected so that none of the components is sequestered by the chelator. In some embodiments, the formulation has an osmolarity that is greater than about 345 mOsol/liter.

As used herein, “formulation” is a preparation or composition in which various components are combined with an active ingredient. As used herein, a formulation may be in the form of an ointment, cream, lotion, gel, salve or the like, for topical application or delivery of the active ingredient to a patient (e.g., a patient in need of the active ingredient). In some embodiments, as appropriate, a formulation is used in conjunction with a delivery system (such as a transdermal patch) impregnated with or containing the formulation and suitable for topical application.

As used herein, by a “patient” is simply a multicellular organism having skin and to whom a formulation as described herein may be applied. The words “subject” and “patient” are used interchangeably. Thus, a patient includes, without limitation, both vertebrate and invertebrate animals. Non-limiting patients include humans, non-human primates (e.g., chimpanzees), laboratory animals (e.g., mice, guinea pigs, rats, rabbits), domesticated animals (e.g., cats, dogs, horses, and pigs)..

By “skin” is meant any surface of the body of a patient containing epithelial tissues including, without limitation, body surfaces with hair follicles including facial skin, skin on the head, skin on the torso, skin on the extremities (e.g., arms and hands, and legs and feet), skin on the palms of the hand, skin on the soles of the feet, skin underneath fingernails and toenails, and mucous membrane-covered body surfaces including those surfaces lining the vagina, the anus, the rectum, the eyes, the ear canal, and the throat. In some embodiments, for hair follicle-containing skin, the skin is shaved or the hair is otherwise removed (e.g., with a depilatory cream) prior to application of a formulation as described herein. In some embodiments, for mucous membrane-covered skin (e.g., covering the surface of the eye), the skin is wiped or dried to reduce the amount of mucous prior to application of a formulation as described herein.

As used herein, by “tissue surface” is meant the surface of a tissue or an organ within a multicellular organism. In some embodiments, the tissue surface comprises epithelial cells. The tissue can be any tissue including, without limitation, a muscle, an organ (e.g., a heart or a kidney) or a diseased tissue (e.g., a solid tumor).

In some embodiments, it may be desirable to retain the integrity of the tissue to which the active ingredient is being topically applied. For example, for a solid tumor, it may be desirable to use the methods and formulations described herein to topically apply (externally to the skin or internally to a tissue surface) an active ingredient (e.g., a chemotherapeutic agent) to the surface of the tumor, where the active ingredient is able to go past the cells at the tumor surface into the core of the tumor, without breaking or permanently damaging any cells at the surface of the tumor. For solid tumors, surgery to remove the tumor may be too risky and invasive, and cutting open the tumor to deliver a drug into the core of the tumor runs the risk that tumor cells may escape the contained solid tumor and metastasize to other points in the body. Yet another benefit of the formulations and methods described herein is the containment of the active ingredient within the tissue or organ that is being treated. Thus, using the formulations and the methods described herein, the active ingredient can be applied to the surface of the organ or tissue without having to permanently damage any cells at the tissue surface.

Of course, as discussed below, the formulations and methods described herein can be used in conjunction with other methodologies that do permanently damage skin cells or cells at the tissue surface. As discussed below, cutting or ulceration can be employed together with the topical application.

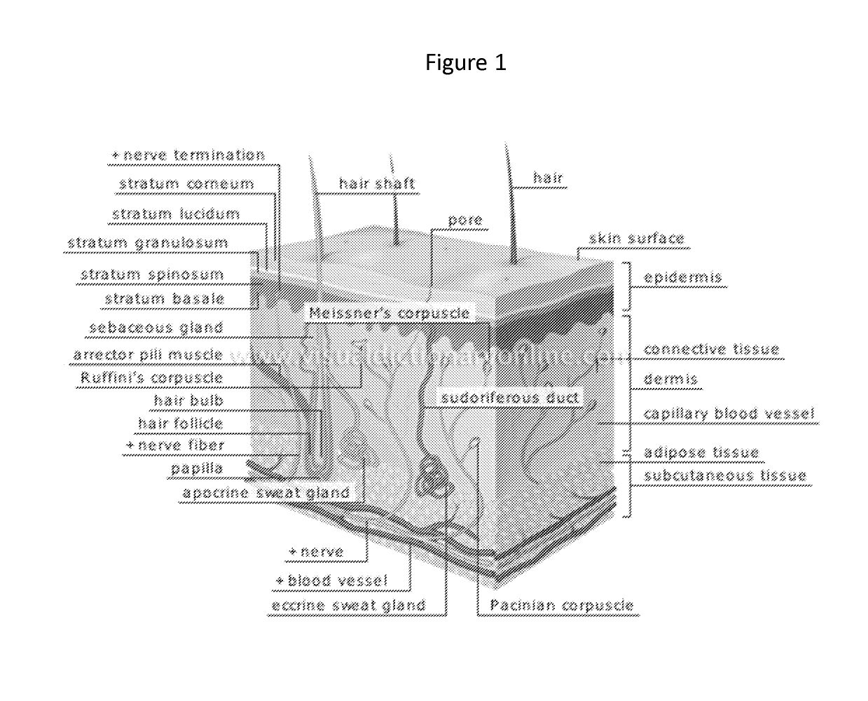

As used herein, by “topical” is meant application of a formulation to skin of a patient or to the surface of the body of the patient, or to the surface of an organ or tissue of a patient. The term “topical” also includes application of a formulation to a mucosal membrane of a patient (e.g., the vagina, eyes, ears, and via the alimentary canal including the mouth, lips, throat. esophagus, stomach, intestines, and anus). For purposes of applying a formulation, topical application to the skin shall include application to the stratum corneum, microinjection to the epidermis (such as can be achieved with microneedles), or use of sonophoresis, iontophoresis or other permeation-enhancing methods, without piercing the basement membrane that separates the epidermis from the dermis and without subsequent injection to the dermis or subcutaneous tissues underlying the dermis (see, e.g., FIG. 1). For purposes of applying a formulation, topical application to a tissue surface of a tissue or an organ shall include application to the surface of the tissue, microinjection to the endothelial cell level at the tissue surface (such as can be achieved with microneedles), or use of sonophoresis, iontophoresis or other permeation-enhancing methods, without piercing the basement membrane that separates the endothelial cell layer from the underlying tissue and without subsequent injection to the tissues underlying the endothelial layer of the tissue or organ.

In some embodiments, the topical formulations described herein are able to facilitate delivery of an active ingredient through the epidermis by carrying the component having an active ingredient activity past the cells of the epidermis and through the basement membrane into the dermis. Components in the formulation facilitate this transdermal or tissue delivery. As described in US Patent Publication No. 2010/0076035 (the entire contents of which are hereby incorporated by reference), if a formulation contains components such that, when the formulation is applied topically, a condition of hypertonicity is created at the topical application site, the hypertonic condition may cause crenation of cells in the skin or in the surface of tissues and organs, which may widen interstitial channels in the skin or tissue surface, or open new channels. These widened and/or newly opened channels in the epidermis allow the component comprising the active ingredient activity in the formulation to be transmitted through the epidermis into the underlying dermis.

As used herein, by an “active ingredient” is meant any component of a formulation that provides pharmacological activity or other direct or contributory effect in the diagnosis, cure, mitigation, treatment, or prevention of disease. An active ingredient may also be referred to as a “drug”. Non-limiting examples of active ingredients that are useful in the topically formulations and methods described herein include antifungal agents; anti-inflammatory agents, such as non-steroidal anti-inflammatory drugs (NSAIDS) and steroidal anti-inflammatory drugs; antibiotics; antiviral agents; anti-neoplastic agents; astringents; anesthetics; systemic drugs; steroid hormones, such as estradiol and testosterone; cosmetic agents, such as skin moisturizers, protectants, and emollients; nutrients, such as vitamins; and ceramides (i.e., a moisture-capturing lipid having a sphingoid based linked to a fatty acid via an amide bond); and other drugs or known to those skilled in the art (e.g., those ingredients listed by the U.S. Food and Drug Agency in “Approved Drug Products with Therapeutic Equivalence Evaluations (Orange Book)”, available at: http://www.fda.gov/Drugs/InformationOnDrugs/ucm129662.htm that are judged suitable by those skilled in the art). In some embodiments, the active ingredient is capable of inducing a desired physiological effect on a targeted skin or other tissue surface (e.g., at the topical application site) other than solely an osmolyte effect, a chelatory effect, or a vasodilatory or vasoconstrictory effect.

Additional active pharmaceutical ingredients include, without limitation, a biological agent, acebutolol, acetaminophen, acetohydoxamic acid, acetophenazine, acyclovir, adrenocorticoids, albuterol, alendronate, allopurinol, alprazolam, alpha hydroxylipids, aluminum hydroxide, amantadine, ambenonium, amiloride, amino acids and amino acid polymers, aminobenzoate potassium, amiodarone HCl, amitriptyline, amobarbital, amlodipine, amoxicillin, amphetamine, ampicillin, amoxapine, androgens, anesthetics, antibody molecules, anticoagulants, anticonvulsants-dione type, antisense molecules, antithyroid medicine, appetite suppressants, aspirin, astemizole, atenolol, atorvastatin, atropine, azatadine, azithromycin, bacampicillin, baclo fen, beclomethasone, belladonna, benfotiamine, benzazepril, bendroflumethiazide, benzoyl peroxide, benzthiazide, benztropine, betamethasone, betha nechol, betaxolol HCl, biperiden, bisacodyl, bisoprolol/HCTZ, bleomycin, botulism toxin, bromocriptine, bromodiphenhydramine, brompheniramine, buclizine, budesonide, bumetanide, bupropion HCl, buspirone, busulfan, butabarbital, butanol, butaperazine, butoconazole nitrate, butorphanol, caffeine, calcitonin, calcium carbonate, camptothecin, capsaicin, captopril, carbamazepine, carbenicillin, carbidopa & levodopa, carbinoxamine inhibitors, carbonic anhydrase, carboplatin, carisoprodol, carotene, carphenazine, carteolol HCl, cascara, cefaclor, cefproxil, cefuroxime, cephalexin, cephradine, cetirizine, chlophedianol, chloral hydrate, chlorambucil, chloramphenicol, chlordiazepoxide, chloroquine, chlorothiazide, chlorotrianisene, chlorpheniramine, chlorpromazine, chlorpropamide, chlorprothixene, chlorthalidone, chlorzoxazone, cholestyramine, cimetidine, cinoxacin, ciprofloxacin, cisapride, cis-platin, clarithromycin, clemastine, clidinium, clindamycin, clofibrate, clomiphere, clonazepam, clonidine, clorazepate, clotrimoxazole, cloxacillin, cloxapine, codeine, colchicine, collagen, coloestipol, conjugated estrogen, contraceptives, corticosterone, cortisone, crornolyn, cyclacillin, cyclandelate, cyclizine, cyclobenzaprine, cyclophosphamide, cyclothiazide, cycrimine, cyproheptadine, cytokines, danazol, darithron, dantrolene, dapsone, daunorubicin, deoxyribonucleic acid, desipramine-HCl, desloratadine, desogestrel, dextroamphetamine, dexamethasone, dexchlorpheniramine, dextromethorphan, diazepan, diclofenac sodium, dicloxacillin, dicyclomine, diethylstilbestrol, diflunisal, digitalis, digoxin, diltiazen, dimenhydrinate, dimethindene, diphenhydramine, diphenidol, diphenoxylate & atrophive, diphenylopyraline, dipyradamole, dirithromycin, disopyramide, disulfiram, divalporex, docusate calcium, docusate potassium, docusate sodium, dopamine, domiphen bromide, doxazosin, doxorubicin, doxylamine, dronabinol, enzymes, enalaprilat, ephedrine, epinephrine, ergoloidmesylates, ergonovine, ergotamine, erythromycins, erythropoietin, conjugated estrogens, estradiol, estrogen, estrone, estropipute, etbarynic acid, ethchlorvynol, ethinyl estradiol, ethopropazine, ethosaximide, ethotoin, etidronate sodium, etodolac, famotidine, felodipine SR, fenoprofen, fenoterol, fentanyl, ferrous fumarate, ferrous gluconate, ferrous sulfate, fexofenadine, finasteride, flavoxate, flecaimide, fluconazole, fluoxetine, fluphenazine, fluprednisolone, flurazepam, fluticasone, fluticasone propionate, fluvastin, fluvoxamine maleate, formoterol fumarate, folic acid, fosinopril, furosemide, gabapentin, ganciclovir, gemfibrozil, glimepiride, glipizide, glyburide, glycopyrrolate, gold compounds, granstronHCl, griseofuwin, growth hormones, guaifenesin, guanabenz acetate, guanadrel, guanethidine, guanfacine, halazepam, haloperidol, heparin, hetacillin, hexobarbital, human growth hormone, hydralazine, hydrochlorothiazide, hydrocodone with APAP, hydrocortisone (cortisol), hydroflunethiazide, hydroxychloroquine, hydroxyzine, hyoscyamine, ibuprofen, imipramine, idebenone, indapamide, indomethacin, isradipine, insulin, interferon, ipratropiumbromide, iofoquinol, iron-polysaccharide, isoetharine, isoniazid, isopropamide, isoproterenol, isosorbide mononitrate S.A, isotretinoin, isoxsuprine, isradipine, itraconazole, ivermectin, kaolin & pectin, ketoconazole, ketoprofen, ketorolac-tromethamine, lactulose, lansoprazole, latanoprost, levodopa, levaflozacin, levonogestrel, levothyroxine, lidocaine, lincomycin, liothyronine, liotrix, lisinopril, lithium, lomefloxacin HCl, loperamide, loracarbef, loratadine, lorazepam, losartan, losartan/HCTZ, lovastatin, loxapine succinate, lymphokines, magnesium hydroxide, magnesium sulfate, magnesium trisilicate, maprotiline, meclizine, meclofenamate, medroxyprogesterone, mefloquine HCl, melatonin, melenamic acid, meloxicam, melphalan, menthol, mephenytoin, mephobarbital, meprobanate, mercaptopurine, mesoridazine, metaproterenol, metaxalone, metformin, metformin hydrochloride, methadone, methamphetamine, methaqualone, metharbital, methenamine, methicillin, methocarbamol, methotrexate, methsuximide, methylchlothinzide, methylcellulose, methyldopa, methylergonovine, methylphenidate, methylprednisolone, methylsergide, methyl salicylate, metformin HCl, metoclopramide, metolazone, metoprolol, metronidazole, mexiletine, miconazole nitrate, minoxidil, misoprostol, mitotane, moclobemide, moexipril HCl, mometasone, monamine oxidase inhibitors, morphine, mupirocin, nabumetone, nadolol, nafazodone, nafcillin, nalidixic acid, naproxen, narcotic analgesics, nedocromil sodium, nefazodone HCl, neomycin, neostigmine, niacin, nicardipine, nicotine, nifedipine, nimodipine, nitrazoxanide, nitrates, nitrofurantoin, nitroglycerin, nizatidine, nomifensine, norethindrone, norethindrone acetate, norfloxacin, norgestimate, norgestrel, nylidrin, nystatin, oflaxacin, omeprazol, orphenadrine, oxacillin, oxaprozin, oxazepam, oxprenolol, oxycodone, oxymetazoline, oxyphenbutazone, pancrelipase, pantothenic acid, papaverine, para-aminosalicylic acid, paramethasone, paregoric, paroxetine, pemoline, penicillamine, penicillin, penicillin-v, pentazocine HCl, pentobarbital, pentokifylline, peptides and peptide fragments, pergolid mesylate, perphenazine, pethidine, phenacetin, phenazopyridine, pheniramine, phenobarbital, phenolphthalein, phenprocoumon, phensuximide, phentolamine mesylate, phenylbutazone, phenylephrine, phenylpropanolamine, phenyl toloxamine, phenytoin, pilocarpine, pindolol, piper acetazine, piroxicum, poloxamer, polycarbophilcalcium, calcium polythiazide, potassium supplements, pravastatin, prazosin, prednisolone, prednisone, primidone, probenecid, probucol, procainamide, procarbazine, prochlorperazine, procyclidine, progesterone, promazine, promethazine, propantheline, propofol, propoxyphene, propranolol, proteins and protein fragments, pruzepam, pseudoephedrine, psoralens, psyllium, pyrazinamide, pyridostigmine, pyrodoxine, pyrilamine, pyrvinium, quinapril, quinestrol, quinethazone, quinidine, quinine, rabeprazole, ramipril, ranitidine, rauwolfia alkaloids, riboflavin, ribonucleic acid, rifampicin, risperidone, ritodrine, salicylates, salmeterol, sannosides a & b, scopolamine, secobarbital, senna, serotonin, sertraline, sildenafil citrate, simethicone, simvastatin, sirtuin inhibitors (such as nicotinamide, AIH, coumarin, sirtinol, alpha-NAD, carbamido-NAD, trichostatin A, suramin sodium, apicidin, BML-210, BML-266, depudecin, HC Toxin, ITSA1, nullscript, phenylbutyrate, sodium, scriptaid, splitomicin, or suberoyl bis-hydroxamic acid), sirtuin activators (such as resveratrol, isonicotinamide, butein, or luteolin), small nucleic acids and nucleic acid fragments (such as aptamers and siRNA), sodium bicarbonate, sodium phosphate, sodium fluoride, sodium nitrate, spironolactone, sucrulfate, sulfacytine, sulfamethoxazole, sulfasalazine, sulfinpyrazone, sulflsoxazole, sulindac, sumatriptan, talbutal, tamoxifen, tamazepam, tenoxicam, terazosin, terbinafine, terbutaline, terconzaole, terfenadine, terphinhydrate, tetracyclines, testosterone and analogs, thiabendazole, thiamine, thioridazine, thiothixene, thonzonium bromide, thyroglobulin, thyroid, thyroxine, tibolone, ticarcillin, timolol, tioconazole, tobramycin, tocainide, tolnaftate, tolazamide, tolbutamide, tolmetin, tramadol, trazodone, tretinoin, triamcinolone, triamterine, triazolam, trichlormethiazide, tricyclic antidepressants, trihexethyl, trifluoperazine, triflupromazine, trihexyphenidyl, trimeprazine, trimethobenzamine, trimethoprim, trimipramine, tripclennamine, triprolidine, troglitazone, trolamine salicylate, tumor necrosis factor, valacyclovir, valproic acid, valsartan, venlafaxine, verapamil, vitamin A, vitamin B-12, vitamin C, vitamin D, vitamin E, vitamin K, voltarin, warfarin sodium, xanthine, zidovudine, zopiclone and zolpidem, and any derivatives of these and combinations of the foregoing. Other active ingredients are listed in U.S. Pat. No. 6,635,274, incorporated herein by reference in its entirety.

In some embodiments, an effective amount of the component comprising an active ingredient activity is present in the formulation described herein or is topically applied in the methods described herein. By “effective amount” is simply an amount of an active ingredient that is effective for whatever the active ingredient is being used for. For example, if the active ingredient has an analgesic activity, the effective amount is simply an amount that can reduce pain in the subject. That amount will of course depend upon the degree of pain, the active ingredient itself (e.g., morphine versus acetaminophen) and the patient (e.g., age, weight, species, etc.). Those of skill can easily adjust the amount of active ingredient in the methods and formulation described herein as appropriate. For example, the concentration of the active ingredient can easily be increased in a formulation by simply reducing the amount of solvent, such as water).

It should be noted that more than one active ingredient may be contained within a formulation as described herein. It should also be noted that an active ingredient (i.e., a component comprising an active ingredient activity) in a topical formulation described herein may serve exclusively as the active ingredient activity provider or it may also serve an addition function. For example, the component comprising an active ingredient activity (e.g., having an antibacterial activity) may also have a vasoactive activity and/or a chelating activity. Certainly, the active ingredient within the formulation may contribute to the total osmolarity of the entire formulation, along with the other components in the formulation, if the active ingredient is soluble in the formulation.

Thus, in some embodiments, the functions of two or more of: the vasoactive agent, the chelator, the active ingredient and the optional components (e.g., the osmolyte and the penetration enhancer) in a single formulation can be provided by a single compound. For example, a bi-functional molecule that combines vasodilation and penetration enhancing properties, such as described in U.S. Pat. No. Published 8,354,116, which is hereby incorporated by reference, can be combined with an osmotic agent and other ingredients as described herein.

Of course, in some embodiments, the vasoactive agent, the chelator, the active ingredient and the optional components (e.g., the osmolyte and the penetration enhancer) in a single formulation are mutually exclusive (or mutually distinct). In other words, the vasoactive agent is not the same as the chelator, and neither is the same as the active ingredient. When the formulation further contains an osmolyte, when the components are mutually exclusive, the vasoactive agent is not the same as the chelator, which is not the same as the active ingredient, which is not the same as the osmolyte. In other words, when components are mutually exclusive, it means that the components are not the same as each other.

The active ingredient (or any other component in the formulation) may not be soluble in the formulation. Rather, the individual components (including the component having active ingredient activity) may be in suspension in the formulation.

In some embodiments, the formulations describe herein further comprise a vasoactive agent. By a “vasoactive agent” is simply a bioactive chemical that can change the vasomotor tone to either increase or decrease blood pressure in the local peripheral area of a blood vessel being treated with the vasoactive agent. Thus, the a vasoactive agent may be a vasoconstrictor or a vasodilator. Multiple vasoactive agents can be combined to result in both rapid and longer-term effects on the skin or tissue surface at the topical application site at which the vasoactive agents are applied.

Vasoconstrictors and vasodilators are well known.

Commonly used vasoconstrictors include, without limitation, antihistiamines, amphetamines, cocaine, caffeine (and other stimulants), psilocybin, tetrahydrozoline HCL, phenylephrine, pseudoephedrine, lysergic acid diethylamide (LSD), ergine (LSA or d-lysergic acid amide), mephedrone, oxymetazoline, epinephrine, ephedrine, adenosine triphosphate, amphetamine, antazoline, asymmetric dimethylarginine, cocaine, dopamine, endothelia, hydroxyamphetamine, isoproterenol, levonordefrin, metaraminol, methamphetamine, methoxamine, methylphenidate, neuropeptide Y, naphazoline, norepinephrine, oxymetazoline, phenylephrine, pseudoephedrine, tetrahydozoline, thromboxane, tramazoline, tyramine, derivatives of these and combinations of the foregoing. A review of topical vasoconstrictors is available at Higgins et al., Laryngoscope 12(12): 422-432, 2011.

Commonly used vasodilators include, without limitation, adrenaline, histamine, prostacyclin, prostaglandin D2, prostaglandin E2, arginine (e.g., L-arginine), nicotinic acid (niacin or vitamin B3), bradykinin, adenosine, heparin, benzyl nicotinate, nitroglycerin, diltiazem, papaverine, tolazoline, and methyl nicotinate. Still additional vasodilators include, without limitation, amrinone, bamethan sulphate, bencyclane fumarate, benfurodil hemisuccinate, benzyl nicotinate, buflomedil hydrochloride, buphenine hydrochloride, butalamine hydrochloride, cetiedil citrate, ciclonicate, cinepazide maleate, cyclandelate, diisopropylammonium dichloroacetate, ethyl nicotinate, hepronicate, hexyl nicotinate, ifenprodil tartrate, inositol nicotinate, isoxsuprine hydrochloride, kallidinogenase, naftidrofuryl oxalate, nicametate citrate, niceritrol, nicoboxil, nicofuranose, nicotinyl alcohol, nicotinyl alcohol tartrate, nitric oxide, nonivamide, oxpentifylline, papaverine, papaveroline, pentifylline, peroxynitrite, pinacidil, pipratecol, propentofyltine, raubasine, suloctidil, teasuprine, thymoxamine hydrochloride, tocopherol nicotinate, tolazoline, xanthinol nicotinate, diazoxide, hydralazine, minoxidil, and sodium nitroprusside. Centrally acting agents include clonidine, quanaberz, and methyl dopa. Alpha adrenoceptor blocking agents include indoramin, phenoxybenzamine, phentolamine, and prazosin. Adrenergic neuron blocking agents include bedmidine, debrisoquine, and guanethidine. ACE inhibitors include benazepril, captopril, cilazapril, enalapril, fosinopril, lisinopril, perindopril, quinapril, and ramipril. Ganglion blocking agents include pentolinium and trimetaphan. Calcium channel blockers include amlodipine, diltiazem, felodipine, isradipine, nicardipine, nifedipine, nimodipine, and verapamil. Prostaglandins including: prostacyclin, thrombuxane A2, leukotrienes, PGA, PGA1, PGA2, PGE1, PGE2, PGD, PGG, and PGH. Angiotensin II analogs include saralasin. Still other suitable vasodilators include nitroglycerin, labetalol, thrazide, isosorbide dinitrate, pentaerythritol tetranitrate, digitalis, hydralazine, diazoxide, and sodium nitroprusside, derivatives of these and combinations of the foregoing. Additional examples of vasodilators include nitroglycerine, arginine and some arginine derivatives, acetylcholine, sodium nitroprusside, methyl nicotinate, hexyl nicotinate, arachidonic acid, prostaglandin D2, prostaglandin 12, tolazoline, papaverine. Arginine is a known substrate for nitric oxide synthase and it is known that nitric oxide can exert a vasodilatory effect.

The vasodilator (or mixture of vasodilators) in the formulation, can be chosen from the classes of endothelium-dependent vasodilators, endothelium-independent vasodilators and prostaglandin-based vasodilators to elicit the production of endogenous prostaglandin. Prodrugs of any of the foregoing vasodilators can also be used. While not wishing to be bound by any particular theory, it may be that inclusion of the vasodilator in the formulation will relax or dilate the dermal arteries and arterioles and therefore increase the volume of blood flow into the capillary network. This increased volume of blood will subsequently result in an increased transcapillary flux of water from the vessel into the surrounding tissue, including the epidermis.

In some embodiments, the vasoactive agent is a locally acting vasodilator. Without wishing to be bound by any particular theory, the vasodilator in the formulations and methods described herein may aid in the penetration of the active ingredient (or the formulation which contains the active ingredient) through the basement membrane which separates the epidermis and the dermis. Once in the dermis the vasodilator acts on the arterioles to induce a transient relaxation of the walls of the blood vessel. This relaxation results in a dilation of the arteriole and therefore an increase of blood volume flow from the arteriole into the dermal capillary bed. The increase in capillary blood volume creates an increase in hydrostatic pressure inside the capillary causing water and plasma to be forced from the capillary into the surrounding tissue. The increase in water and plasma in the interstitial spaces of the surrounding tissue moves the components of the formulation (e.g., the active ingredient) through the tissue with the flow of the fluid. The increased volume in the interstitial spaces in the tissue as a result of the action of the chelator in combination with the action of the vasodilator allows more of the active ingredient to move through the epidermis and through the basement membrane and into the dermis more readily than without the presence of other components of the formulation (e.g., the component with chelating activity). This increase in drug movement into the deep tissues (e.g., below the dermis) and/or into either the lymphatic or blood circulatory system creates a greater bioavailability of the active ingredient to the patient for the potential of a greater biological or medical effect.

Without wishing to be bound by any particular theory, the presence of a vasoactive agent and chelator in the same formulation (e.g., a formulation also containing an active ingredient) may allow the chelator and the vasoactive agent to act together synergistically in an unexpected manner. For example, the chelator can weaken the inter-cellular barriers (e.g., tight junctions between cells formed by protein-protein interactions of cell surface proteins on two adjacent cells), allowing the vasoactive agent within the formulation (together with the active ingredient) to enter the interstitial space. As the vasoactive agent (e.g., a vasodilator) thus gains access to move through the interstitial spaces to reach the dermis and the underlying blood vessel, fluid is released by the vasodilator (by dilating the blood vessel, such an arteriole), releasing plasma into the surrounding tissue. That plasma flows into the interstitial space widened by the chelator, thus allowing more of the formulation containing the chelator, active ingredient, and vasoactive agent, to penetrate into the tissue. By this sequential process, the delivery of the active ingredient through the surface (e.g., skin surface or tumor surface) into the underlying tissue is enhanced. Additionally, if an osmolyte is present in the formulation, the osmolyte may also facilitate the widening of the interstitial space by causing the crenation of the affected cells lining the interstitial space, thereby increasing the volume of the interstitial space.

In some embodiments, application of the formulation can cause an increase in blood flow at or near the region of application. The increase can be greater than or equal to 1%, 5%, 10%, or more. The increase in blood flow can be measured relative to blood flow prior to treatment with the formulation or relative to blood flow in skin treated with a control formulation lacking the vasoactive agent. The increased blood flow may be measured using laser Doppler velocimetry, which typically outputs a voltage that is proportional to the velocity of cells moving through the blood. Such measurements are known in the art (see, e.g., Holloway G A Jr, Watkins D W., 1977, Laser Doppler measurement of cutaneous blood flow. J Invest Dermatol., September; 69(3):306-9). The test can be performed on participants after a 20-minute acclimatization period in a warm environment (room temperature 24° C.). For each subject, the blood flow response is measured with the non-invasive test before and after the application of the test formulation and at various intervals of time after the application until the blood flow has returned to a pre-application level. The measurement of skin or tissue surface blood flow can be evaluated using a Laser Doppler Perfusion Imager (LDPI Lisca 2.0, Lisca development AB, Linkoping, Sweden). This apparatus employs a 1 mW Helium-Neon laser beam of 633 nm wavelength, which sequentially scans the tested area. Typically, maximum number of measured spots is 4096 and the apparatus produces a color-coded image of the tissue perfusion distribution on a computer monitor. The data acquired from the instrument can be statistically analyzed with The Minitab statistical package (Minitab, State College, Pa.) for personal computers. For intra-group comparisons, the paired t-test can be used to compare changes between baseline and the maximal vasodilation. The test can be used for comparison between the two groups of patients. Changes in the microvascular blood flow can be expressed as the difference between the peak response and the baseline blood flow (e.g., in ml/min, laser-Doppler velocimetry voltage readout, or other suitable units). This increased blood flow can enhance the penetration of an active ingredient into or through the skin or tissue surface.

Of course, more than one vasoactive agent may be contained within a formulation as described herein. It should also be noted that a vasoactive agent (e.g., a vasodilator) in a topical formulation described herein may serve exclusively as the vasoactive activity provider or it may also serve an addition function. For example, the vasoactive agent (e.g., the vasodilator) may also have a chelating activity, or may act as an active ingredient. For example, niacin (nicotinic acid) has a vasodilating activity but it can also serve as an active ingredient for its other properties (e.g., niacin has lipid lowering and anti-atherosclerotic properties). Certainly, the vasoactive agent within the formulation may contribute to the osmolarity of the formulation, along with the other components in the formulation, if the vasoactive agent is soluble in the formulation.

In some embodiments, the formulation comprises a chelator. A “chelator” or a “chelating agent” is a chemical compound that, in the presence of an ion with a valency higher than monovalency (e.g., a divalent cation or a divalent metal cation), binds to that ion and sequesters it, effectively trapping that ion and making it unable to interact with other molecules. Typical ions having valency higher than monovalency that will be bound by and sequestered by a chelator include Ca2+ and Mg2+. Note that a chelator, as used herein, will not bind and sequester a monovalent cation such as Na+.

It should be noted that the other components in the formulations described herein are selected so that they are not sequestered by the chelator. By “sequestered” is meant a chelator binds to and holds an ion having a valency higher than monovalency (e.g., holds a divalent cation) such that the bound ion is unable to freely move and function in the formulation. For example, if an osmolyte is present in the formulation, the osmolyte is not a divalent ion (or an ion with a higher valency) because it may be sequestered by the chelator and thus will not be able to function as an osmolyte in the formulation.

Two adjacent cells in a patient (e.g., in the patient skin or on the surface of an organ) are held tightly against one another by multiple bridges formed by cell surface molecules on both cells. Divalent ions/divalent cations (e.g., Ca²⁺, Mg²⁺) are often essential in the bridge formed by two cell surface molecules on adjacent cells. Indeed, if the divalent ion is not present, the bridge may not form. By including a chelator in a formulation comprising an active ingredient (or by applying a chelator together with or sequentially with an active ingredient), the chelator will sequester divalent ions in the interstitial spaces, reversing and preventing the formation of inter-cellular bridges. Moreover, without wishing to be bound by any particular theory, as the divalent ions in the interstitial spaces are subject, like any solute, to an equilibrium gradient in the solution, when more free divalent ions are removed from the extracellular fluid in the interstitial space, those divalent ions complexed in bridges joining two adjacent cells may become uncomplexed, breaking that bridge. The cycle of breaking bridges between two adjoining cells and sequestering the divalent ions will continue. However, because the integrity of the cell itself is not compromised, the cell is not permanently damaged by the formulation (or application method) described herein.

Non-limiting examples of components comprising a chelating activity include BAPTA (1,2-bis(o-aminophenoxy)ethane-N,N,N′,N′-tetraacetic acid), Fura-2 (see Grynkiewicz et al., J. Biol. Chem. 260(6) 3440-3450, 1985), DMSA (dimercaptosuccinic acid), ALA (alpha lipoic acid), DMPS (2,3-dimercapto-l-propanesulfonic acid), deferoxamine, deferasirox, dimercaprol, penicillamine, EDTA (ethylenediaminetetraacetic acid), EGTA (ethylene glycol tetraacetic acid), and ethylenediamineacetate. Additional chelating agents are described in U.S. Pat. No. 4,528,196 (incorporated by reference)

In some embodiments, the component with chelating activity is a divalent cation chelator. EGTA, EDTA and CDTA are non-limiting examples of divalent cation chelators. The divalent cation chelator in the formulation is present to physically separate the calcium, magnesium, and manganese as well as other divalent cations from the protein-protein bonds present in the interstitial spaces created by like-ectoproteins protruding from each adjacent cell. The removal of the divalent cation from these protein-protein interactions physically breaks the bonds holding the two adjacent cells together in the native position. The breaking of these protein-protein bonds then allows for the interstitial spaces to transiently expand, lowering the barriers to movement in the skin tissue (or other tissue at the surface of a tissue or organ) for the active ingredient (e.g., a therapeutic or diagnostic agent). As this separation of the protein-protein bond is transient, the skin cells or cells at the tissue surface are not permanently damaged by contact with the formulation comprising the chelator.

It shall be understood that more than one chelator may be contained within a formulation as described herein. It should also be noted that a chelator in a formulation described herein may serve exclusively as a chelating activity provider or it may also serve an addition function. For example, the chelator (e.g., a divalent cation chelator) may also have a vasoactive activity, or may act as an active ingredient. Certainly, the chelator within the formulation may contribute to the osmolarity of the formulation, along with the other components in the formulation, if the chelator is soluble in the formulation.

Without wanting to be bound by any particular mechanistic hypothesis, the hypertonicity of the formulation can cause crenation of cells in the skin or in the tissue surface at the site that the formulation is applied. This crenation can widen or open interstitial spaces in the skin or tissue surface. The component with vasoactive activity (e.g., a vasodilator) in the formulation can act on the microvasculature within the dermis to generate a vasodilation event that releases plasma and/or interstitial fluid into the interstitial spaces and extracellular spaces surrounding the vessel and into the dermis and epidermis. Thus, the high osmolarity of the formulation allows an increase in the intracellular osmotic pressure of the skin cells or cells at the tissue surface at the application site of the formulation. This increase in intracellular osmotic pressure will move water from inside the cell to the interstitial space. This action will in turn cause a decrease in the volume of the cell and conversely, increase the volume and size of the interstitial spaces.

Osmolarity must be understood in terms of two additional art-known terms, namely “isotonic” and “hypertonic,” both of which refer to tonicity (or tone) in two or more fluids. Two fluids are said to be isotonic (or isosmotic), when they have equal tension or tone. For example, an extracellular solution that is isotonic to the cytoplasm of a cell will have the same tonicity as the cell and thus no net flow of water will cross the cell membrane. On the other hand, the term “hypertonic” means that a given fluid has a greater degree of tone or tension, and thus a higher osmotic pressure (i.e., water pressure flow across a biological membrane) relative to another fluid. For example, if an extracellular fluid has greater amounts of solutes than the cytoplasm of a cell, the extracellular fluid is said to be hypertonic to the cell, and water will flow out of the cell into the extracellular fluid in an attempt to dilute the solutes in extracellular fluid and thus reduce the tension between the extracellular fluid and the cytoplasm. This flow of water out of a cell will cause the cell to shrink or undergo “crenation” while the extracellular space expands with the additional water flowing into it from the shrinking cells.

Hypertonicity and isotonicity can be determined by measuring and calculating the osmolarity of the ingredients in the topical formulation and comparing the osmolarity of the formulation to the physiological osmolarity of a subject. All of the components in the formulations described herein can contribute to the osmolarity of the formulation because, once in the formulation, the components are solutes within that formulation. The definition of osmolarity is as follows: Osmolarity (mOsm/L)=Σconcentrations of all solutes (mMoles/L). An osmole (Osmol) is a unit of osmotic pressure equivalent to the amount of solute that dissociates in solution to form one mole (Avogadro's number) of a non-dissociable substance (e.g., an atom or a compound such as glucose). If the calculated osmolarity of the formulation is greater than the physiological osmolarity of the subject, then the formulation is said to be hypertonic to the subject. If the calculated osmolarity of the formulation is the same as the physiological osmolarity of the subject, then the formulation is said to be isotonic to the subject. Note that if a formulation has an osmolarity that is lower than the physiological osmolarity of the subject, the formulation is said to be hypotonic to the subject.

There is an accepted range of osmolarity of vertebrate subjects defined as isotonic ranging from 240-340 milliOsmoles/Liter with a tighter range of 280-310 milliOsmoles/Liter. In other words, any formulation that has an osmolarity value of greater than 345 milliOsmoles/Liter (or 345 mOsM) will be considered to be hypertonic to a vertebrate subject.

When the osmolarity of the entire formulation is considered, the osmolar value is calculated for each of the components in the formulation individually, and the total osmolarity of the formulation determined by adding the osmolarity of the individual components.

Using the known osmolarity of vertebrate subjects, some commonly used isotonic, physiologically acceptable formulations are as follows.

TABLE 1
Isotonic Formulations

| Formulation | Calculation of Osmolarity |
|---|---|
| 5% Glucose | 5% Glucose (w/w) |
| (see Nette et al, | Glucose Molecular weight = 180.16 |
| Nephrol. Dial. | 5% glucose (w/w) = 50 g/liter |
| Transplant 17: | Since a glucose molecule does not further |
| 1275-1280, 2002) | dissociate in solution, 50 grams/liter divided by |
| | 180.16 g/mol, which equals 0.277 moles/liter or |
| | 277.5 mM, which equals 277.5 mOsmol/liter |
| 4.5% Sorbitol | Sorbitol Molecular weight = 182.17 |
| (see Jumaa and | 4.5% Sorbitol (w/w) = 45 g/liter |
| Muller, Eur. J. of | Since a sorbitol molecule does not further |
| Pharm. Sciences 9: | dissociate in solution, 45 grams/liter divided by |
| 207-212 (1999) | 182.17 grams/mole equals 0.247M or 247 mM, or |
| | 247 mOsmol/liter |

Note that the calculation of osmolarity of the formulations in Table 1 is a simple matter because there is only a single component to be calculated (since the osmolarity of water is negligible).

However, when the formulation has multiple components, the osmolarity of the entire formulation is calculated by adding the osmolarity amounts of each component. For example, a formulation that contains 5% glucose and 4.5% sorbitol in water will have a total osmolarity of 524.5 mOsM (which is the sum of 247 mOsM from 4.5% sorbitol plus 277.5 mOsM from 5% glucose).

A component whose main function in a formulation is to raise the osmolarity of the formulation may be referred to as an “osmolyte.” In some embodiments, an osmolyte is a molecule having an affinity for water (i.e., hydrophilicity or hygroscopicity). When present in a pharmaceutical formulation, an osmolyte is able to draw water from cells, vasculature, or other structures of the body (e.g., from the skin). Because of the presence of a chelator in the formulation, when an osmolyte is also present in that formulation, and that osmolyte is an ion, that ionic osmolyte cannot have a valency higher than a monovalency. For example, when an osmolyte is present in a chelator-containing formulation, the osmolyte cannot be a divalent cation (or a trivalent or quadvalent cation), because the chelator will complex with the divalent cation (or trivalent or quadvalent cation) and effectively sequester it and prevent is ability to function as an osmolyte. Some non-limiting osmolytes that may be used in the formulations described herein include sorbitol, and glucose. In addition to sorbitol, and glucose, some other common physiologically acceptable osmolytes include (but are not limited to) sugar osmolytes such as monosaccharides (e.g., mannitol, galactitol, fucitol, iditol, inositol, glucose, fructose, galactose, ribose, rhamnose, and xylopyranose), disaccharides (e.g., maltitol, lactitol, isomalt, sucrose, lactose, maltose, trehalose, cellobiose, gentiobiose, isomaltose, kojibiose, laminaribiose, marmobiose, melibiose, nigerose, rutinose and xylobiose), and monovalent ions (such as lithium, sodium, and potassium). Note that a monovalent ion osmolyte may be contributed by a salt comprising that monovalent ion (e.g., NaCl providing the Na+ monovalent ion osmolyte).

Note that when an osmolyte is an ion, the valency of the ion cannot be above monovalency, or the “greater valency than monovalency” ion osmolyte will be effectively rendered nonfunctional because it may be bound by and sequestered by the chelator in the formulation. By “valency” is meant the number of valence electrons available in the ion. Thus, monovalent ions have one electron available, such as one cation (e.g., Na+, K+, and Li+) or one electron (e.g., Cl— or F—).

In further embodiments, the formulation described herein comprises a lipid component. The presence of a lipid component in the formulation may facilitate the movement of the other components through the layers of the stratum corneum and to the interface with the stratum corneum and the other layers of the epidermis. The lipid component may be in the form of a lipid-enriched stable pharmaceutical base of the formulation. See Alvarez and Rodriguez “Lipids in Pharmaceutical and Cosmetic Preparations,” Grasas y Aceites 51: 74-96, 2000. Non-limiting lipids that can be used include simple lipids such as vegetable oil lipids (e.g., soybean oil, olive oil, safflower oil), animal oils (e.g., fish oil), fats (e.g., shea butter), wax (e.g., bees wax, lanolin), and compound lipids such as phospholipids (e.g., diphosphatidyl glycerols, phosphatidyl cholines, phosphatidyl serines, phosphatidyl inosiols, phosphatidic acids, phosphatidyl glycerols, and phosphine analogs), sphingolipids (e.g., sphingophospolipids and sphingoglycolipids), glycolipids, and sulfolipids, and derived lipids such as fat-soluble vitamins (e.g., vitamin A, vitamin D, vitamin E, and vitamin K), prostaglandins (e.g., PGA2, PGB2, etc.), and steroids including sterols and sterol esters (e.g., cholesterol), sterylglycosides and acylsterylglycoside, sterol sulfates, and bile acids and their conjugates.

In further embodiment, the formulation described herein comprises a a penetration enhancer. By “penetration enhancer” is meant a compound, particle, or other substance or material that when included in a formulation that is applied topically to the skin or to the tissue surface, increases the rate or amount of transport of an active ingredient in the formulation past the cells (living or dead) of the epidermis. Non-limiting examples of penetration enhancers include individual fatty acids, fatty acid esters, polyols, amides, various anionic, cationic and nonionic surfactants such as but not limited to sodium laurate and sodium lauryl sulfate, phospholipids, cholesterol and cholesterol derivatives, m-pyrrole, dimethyl acetamide, limonene, sphingolipids, ceramides, terpenes, alkanones, menthol, various organic acids, such as but not limited to salicylic acid, citric and succininc acid, prostaglandin, decyl methyl sulfoxide, urea, sulfoxide alcohols, plant extract oils. Suitable fatty acids include without limitation: linoleic acids, linolenic acids, oleic acids, stearic acids, and myristic acids. Phospholipids include without limitation: phosphatidylcholine, phosphatidylethanolamine, and phosphatidylserine. Plant extract oils include oils of peanut, hemp, borage, olive, sunflower, soybean, monoi and macadamia. The plant extract oil can be mixed with an alcohol such as ethyl alcohol, isopropyl alcohol, and methyl alcohol.

In further embodiments, the formulation is applied with a transpiration barrier. A “transpiration barrier” shall mean a component such as a solid patch, a hydrophobic chemical component, or a self-assembling chemical component (including components that form gels) that is capable of preventing water loss from skin or tissue surface due to transpiration when applied to the skin or tissue surface of a patient. If a transpiration barrier is used, the pressure created by the released plasma can build at the impermeable transpiration barrier to create a second osmotic pressure event based on water influx from the vasculature. The first osmotic event upon application of a formulation described herein (e.g., containing an active ingredient, a vasoactive agent, a chelator, and optionally an osmolyte) is a subtle crenating process that can open up the epithelium (by widening the interstitial spaces between the cells in the epidermis) for better drug and particle movement. The second event is the creation of a bolus-type gradient of hydrodynamic pressure in the localized skin tissue or tissue surface tissue (e.g., the surface of the left ventricle of the heart) due to the presence of excessive amounts of interstitial fluid released from the microvasculature by the vasodilator with no place to go except to be re-directed back into the body.

In some embodiments, the formulation can also include solvents, excipients, preservatives, skin conditions, emulsifiers, carriers, polymers, thickeners, phospholipids, fatty acids, cholesterols, complex lipids, prostaglandins, vitamins and vitamin derivatives, antioxidants, humectants, surfactants. Other components may be included in the pharmaceutical preparation that promote passive dermal penetration of chemicals and pharmaceuticals, including urea, organic solvents, such as dimethyl sulfoxide (DMSO), and others. Yet additional components include excipients or carries such as, without limitation, water, Stearyl Alcohol, Polysorbate 20, Caprylic/Capric Glyceride, Petrolatum, Beeswax, Lecithin, Dimethicone, Alkylmethyl Siloxane, Stearic Acid, Palmitic Acid, Lanolin, Linoleic Acid, Isopropyl Myristate, Stearyl Octanoate and Cetyl Octanoate, and Polysorbate 80.

The solvent may be polar, non-polar, aqueous, non-aqueous, organic or inorganic in composition. Common solvents include, without limitation, water and propylene glycol.

Of course, as the skilled practitioner understands, the addition of extra components to a formulation as described herein depends largely upon the form of the formulation. The formulation may be in the form of a lotion, cream, gel, paste, nanoparticle powder, spray, aerosol, or milk. Moreover, different components of the formulation may be encapsulated in a simple or complex lipid mixture for physical separation from the other component parts of the formulation, which may not be compatible with each other or for which there is a need to enhance the lipid-characteristics of the component to facilitate transmigration through the stratum corneum. Methods for encapsulating a component in a simple or complex lipid mixture are known (see, e.g., U.S. Pat. Nos. 7,867,981 and 6,406,713, both incorporated herein by reference).

As a result of using the formulations and/or application methods described herein, an expanded range of candidate transdermal active ingredients can be used. For example, by using such formulations, higher molecular weight and less hydrophobic active ingredients can be transdermally delivered.

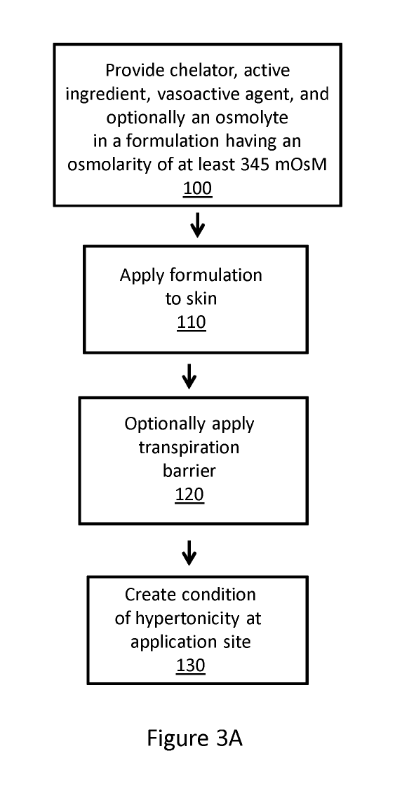

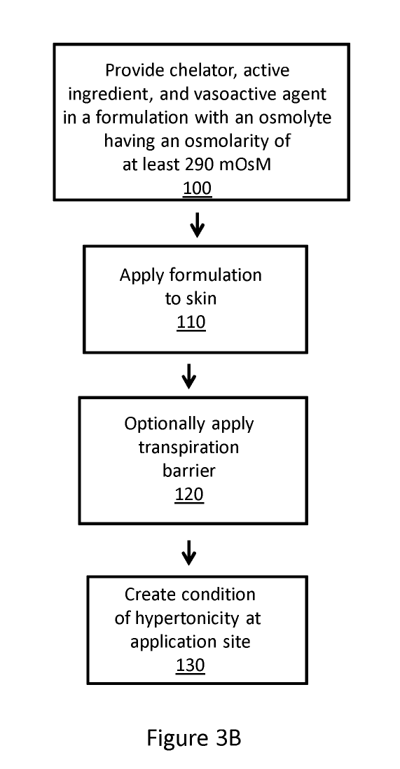

FIG. 3A provides a flow diagram outlining the steps of another non-limiting method for transdermal delivery of an active ingredient to a patient. The transdermally delivered active ingredient penetrates through the epidermis into the dermis and optionally into the systemic circulation of the patient, for example, via the blood or lymphatic system. In the method depicted schematically in FIG. 3B, the active ingredient is combined in a formulation together with a component having vasoactive activity (e.g., a vasodilator or vasoconstrictor), a component having chelating activity, and, optionally, a component having osmolyte activity. In the method depicted in FIG. 3A, the osmolarity of the collective components of the formulation is at least about 345 mOsM. In some embodiments, the osmolarity of the collective components of the formulation is at least about 340 mOsmoles/liter, or at least about 350 mOsmoles/liter, or at least about 375 mOsmoles/liter, or at least about 400 mOsmoles/liter, or at least about 450 mOsmoles/liter, or at least about 500 mOsmoles/liter, or at least about 600 mOsmoles/liter, or at least about 750 mOsmoles/liter, or at least about 900 mOsmoles/liter. This level of osmolarity can be achieved either by the components in the formulation (i.e., the component with chelating activity, the component with active ingredient activity, and the component with vasoactive activity), or by optional the addition of a compound comprising an osmolyte activity specifically added to increase the osmolarity of the formulation. Indeed, the sum of all of the components of the formulation (whatever additional function those components may provide) may contribute to the osmolarity of the formulation.

FIG. 3B provides a flow diagram outlining the steps of a non-limiting method for transdermal delivery of an active ingredient to a patient. As in the method of FIG. 3A, the transdermally delivered active ingredient penetrates through the epidermis into the dermis and optionally into the systemic circulation of the patient, for example, via the blood or lymphatic system. The active ingredient is combined in a formulation with a component having vasoactive activity (e.g., a vasodilator or vasoconstrictor) and a component having chelating activity, and a component having osmolyte activity, where the component having osmolyte activity has an osmolarity of at least about 290 mOsM. In some embodiments, the osmolarity of the component having osmolyte activity (e.g., sorbitol) has an osmolarity of at least about 300 mOsM, or at least about 310 mOsmoles/liter, or at least about 320 mOsmoles/liter, or at least about 330 mOsmoles/liter, or at least about 340 mOsmoles/liter, or at least about 350 mOsmoles/liter, or at least about 400 mOsmoles/liter, or at least about 450 mOsmoles/liter, or at least about 500 mOsmoles/liter, or at least about 550 mOsmoles/liter, or at least about 600 mOsmoles/liter.

Optionally, the formulation may include excipients, solvents, penetration enhancers, lipids, or other components. The formulation can also be a patch or a component of a patch or similar drug delivery device.

The formulation can be applied to the skin or tissue surface; i.e., topically (step 110). For example, the formulation can be a cream, lotion, ointment, gel, or other substance suitable for topical application to the skin or tissue surface. Optionally, the skin or tissue surface can be worked to enhance the penetration of the active ingredient past the epidermis (e.g., into or through the basement membrane). Various methods of working skin are known. For example, the skin or tissue surface may be mechanically worked in the form of massaging or sonophoresis (e.g., via ultrasound) which can exert mechanical work and enhance penetration. The skin or tissue surface may also be worked by electrical work such as iontophoresis.

Skin or tissue surface working processes that permanently damage cells may also be used, as long as the formulation itself does not cause the permanent damage. For example, the skin or tissue surface can be worked by cutting, ulceration, wound formation or piercing. For example, piercing the skin with microneedles (e.g., with a device having projections designed to pierce the stratum corneum without the substantial triggering of deeper pain receptors) can aid in the transdermal delivery process of the active ingredient. Microneedles are disclosed, for example, in U.S. Pat. No. 6,611,707, which is incorporated herein by reference in its entirety. Other methods of working the skin and tissue surfaces are commonly known.

In some embodiments, the formulation is delivered into the skin or into the tissue surface. For example, for delivery to the skin, the formulation can be injected into the epidermis with microneedles. For delivery to a tissue surface (e.g., the surface of a liver), the formulation can be injected into the endothelial cells covering the surface of the liver with microneedles. In some embodiments, the method for delivery of an active ingredient using the formulations and methods described herein includes optionally applying the formulation with a transpiration barrier (step 120 in FIGS. 3A and 3B). The transpiration barrier can be a water impermeable drug administration patch; for example, a sheet of water-resistant plastic with an adhesive layer or other attachment mechanism (e.g., a bandage). The patch can be applied atop a formulation applied to the skin or tissue surface. Alternately, the patch can be impregnated with the formulation and applied to the skin or tissue surface to contact the vasoactive agent, active ingredient, and osmolyte with the skin or tissue surface while forming the transpiration barrier. A water-impermeable wrap, glove, sock, mitten, or the like can also serve to create a physical barrier. Alternately, or in addition, the transpiration barrier can include a molecular (i.e., chemical) barrier; i.e., one that contains a plurality of molecules or particles that are at least initially unbonded and which dry on or embed in the skin or tissue surface to produce a moisture-resistant barrier. For example, the molecular barrier can include silicone, titanium oxide, polyvinyl alcohol and hydrogels. It should be noted that both a chemical barrier and a physical barrier can be used together or sequentially. In another embodiment, a water-resistant patch is applied to the skin or tissue surface for a period (e.g., 0.5 to 60 minutes) prior to removal of the patch and application of a formula described herein.

By including, in the formulation, one or more components (e.g., a component with a chelating activity and a component with an active ingredient activity) at a high enough concentration, a condition of hypertonicity can be created in the skin or tissue surface local to the area at which the formulation is applied (step 130 in FIGS. 3A and 3B). The hypertonic condition can include an elevated osmotic pressure in the extracellular milieu as compared to the intracellular cytoplasm of the skin cells or of the cells (e.g., endothelial cells) at the tissue surface. This condition of hypertonicity can work cooperatively or synergistically with the other activities provided by the components in the formulation (e.g., the vasoactive activity and/or the chelating activity) to enhance delivery of the active ingredient into and through the epidermis, and/or into and through the basement membrane to the dermis or other tissue underlying the epidermis. In some embodiments, an active ingredient in the formulation that is delivered into and through the epidermis can enter the systemic circulation via the blood system or the lymphatic system.

The high osmolarity of the formulation can continue to exert its effect on cells as the components of the formulation move through the epidermis, compounding or synergizing the effect of the component with vasodilating activity on the movement of the active ingredient within the epidermis and the dermis. The combination of the active ingredient, chelator, vasoactive agent and, optionally, the osmolyte, where the osmolarity of the formulation as a whole is greater than 345 mOsM or, if the osmolyte is present, the osmolarity of the can generate a larger gradient than osmotic pressure generated from the epidermal cell water movement alone and can induce greater physical space between the epithelial cells in which drug molecules can move. This combined and elevated osmotic pressure can continue to drive the active drug ingredient through the basement membrane and into the dermis to deliver the active agent to local dermal or subcutaneous tissues or to the lymph and blood capillaries for systemic distribution.

In accordance with illustrative embodiments of the present invention, a formulation containing an active ingredient that also includes a vasoactive agent and a chelator which can work together in an additive or synergistic manner to enable penetration of the active agent into the skin (epidermis or dermis) or through the skin and into general systemic blood circulation thereby to exert a local or systemic therapeutic effect, respectively. Optionally, the formulation includes an osmolyte. Optionally, a transpiration barrier, penetration enhancer, or both can increase the effectiveness of the penetration, also in an additive or synergistic manner. As a result of using such formulations, an expanded range of candidate transdermal active ingredients can be used. For example, by using such formulations, higher molecular weight and less hydrophobic active agents can penetrate the epithelial tissue.

In an illustrative embodiment, the formulation containing a chelator, an active ingredient, and a vasoactive agent is applied to the skin and the vasoactive agent is delivered to the dermis. As a result, the vasoactive agent contacts the cutaneous vasculature. As a result of contact with the vasculature, the vasoactive agent can increase blood flow in the skin (e.g. by greater than 1%, 5% or 10%). The increase in blood flow can be measured relative to blood flow prior to treatment with the formulation or relative to blood flow in skin treated with a control formulation lacking the vasoactive agent. The increased blood flow can be measured using laser Doppler velocimetry, which typically outputs a voltage that is proportional to the velocity of cells moving through the blood. When an osmolyte is included in the formulation, together with osmotic effects of the osmolyte on the tonicity of the skin, this increased blood flow can enhance the penetration of an active ingredient into or through the skin. In a further illustrative embodiment, such formulations are used to enhance the uptake of anti-neoplastic active ingredients into a skin lesion or tumor. The anti-neoplastic active ingredient can, for example, act on the sirtuin pathway.

When the formulation is applied topically to a region of the epidermis, the chelator, active ingredient, vasoactive agent, and optional osmolyte and transpiration barrier assist the vasoactive agent in crossing the epidermis and entering the dermis. In the dermis, the vasoactive agent acts on the microcirculatory system to increase blood flow in the skin. An increase or decrease in blood flow in the local dermis surrounding the area of formulation application will reflect the increased or decreased permeation of fluid through the blood vessels in the skin of the patient. As a result of the increased blood flow, and possibly in connection with the optionally present osmolyte and/or transpiration barrier, the active ingredient is transported into the dermis, and possibly into systemic circulation.

A formulation can be tested for its ability to increase circulation using laser Doppler velocimetry measurements. Such measurements are known in the art (see, e.g., Holloway G A Jr, Watkins D W., 1977, Laser Doppler measurement of cutaneous blood flow. J Invest Dermatol., September; 69(3):306-9).The test can be performed on participants after a 20-minute acclimatization period in a warm environment (room temperature 24° C.). For each subject, the blood flow response is measured with the non-invasive test before and after the application of the test formulation and at various intervals of time after the application until the blood flow has returned to a pre-application level. The measurement of skin blood flow can be evaluated using a Laser Doppler Perfusion Imager (LDPI Lisca 2.0, Lisca development AB, Linkoping, Sweden). This apparatus employs a 1 mW Helium-Neon laser beam of 633 nm wavelength, which sequentially scans the tested area. Typically, maximum number of measured spots is 4096 and the apparatus produces a color-coded image of the tissue perfusion distribution on a computer monitor. The data acquired from the instrument can be statistically analyzed with The Minitab statistical package (Minitab, State College, Pennsylvania) for personal computers. For intra-group comparisons, the paired t-test can be used to compare changes between baseline and the maximal vasodilation. The test can be used for comparison between the two groups of patients. Changes in the microvascular blood flow can be expressed as the difference between the peak response and the baseline blood flow (e.g., in ml/min, laser-Doppler velocimetry voltage readout, or other suitable units).

In some embodiments of the invention, application of the formulation can cause an increase in blood flow at or near the region of application. The increase can be greater than or equal to 1%, 5%., 10%, or more.

In some embodiments, the formulation comprising an active ingredient, a component having chelating activity, and a component having vasoactive activity is hygroscopic. In some embodiments, the formulation is packaged in a water resistant container.

Although convenient, it is not necessary to include all of the aforementioned components of the formulation in a single composition. Rather, the various components can be applied sequentially and in various orders to the skin of a patient so long as the ultimate result is to combine the component with the active ingredient activity, the component with the vasoactive activity, and the component with the chelating activity on the skin at sufficient concentration of each component so that the total osmolarity of the combined component is hypertonic to the patient (i.e., greater than about 340 mOsM) to effect penetration of the active ingredient to therapeutically effective levels.

Similarly, a transpiration barrier can also be applied sequentially with respect to the other components one or more times.

Table 2 below provides a non-limiting list of combinations of components and application methods that can employed for the transdermal delivery of a component with an active ingredient activity.

TABLE 2

| | Activity of Components | Application Method |
|---|---|---|
| Combination | (in a Solvent) | or Purpose |
| 1 | Chelating activity | Simultaneous |
| | Active ingredient activity | |
| | Osmolyte | |
| 2 | Chelating activity | Simultaneous |
| | Active ingredient activity | |
| | Vasodilating activity | |
| 3 | Chelating activity | Simultaneous |
| | Active ingredient activity | |
| | Vasodilating activity | |
| | Osmolyte activity | |
| 4 | Chelating activity | Simultaneous |
| | Active ingredient activity | |
| | Penetration Enhancer activity | |
| | Vasodilating activity | |
| | Osmolyte activity | |

The non-limiting combinations set forth in Table 2 can of course be modified. For example, they can all be used with two or more active ingredients and/or they can all be used with two or more chelators (e.g., each with different ionic avidities). When there is a vasodilator present (e.g., in combinations 4, 6, and 7), one or more additional vasodilator can also be employed. These combinations 4, 6, and 7 (and others) can be used to treat disease or used to modify disease processes. Additionally, the combinations may be used to deliver an active ingredient for diagnostic procedure. For example, a fluorescent dye may be used as an active ingredient and delivered transdermally to a specific site (e.g., to mark tissue damage at a site of traumatic injury).

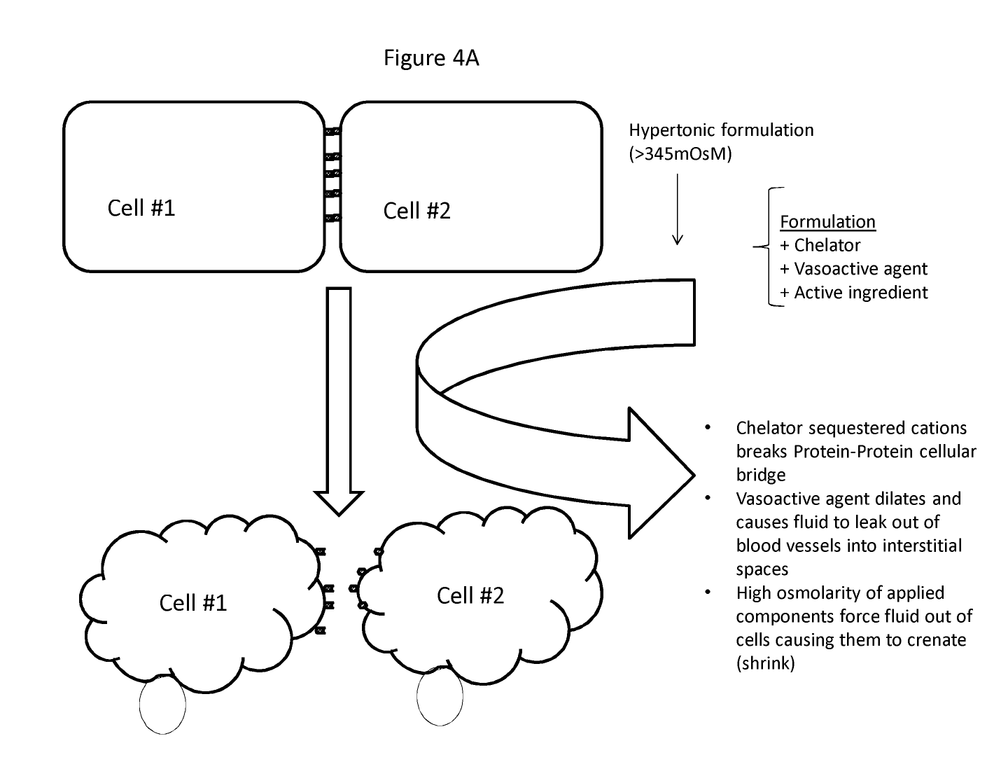

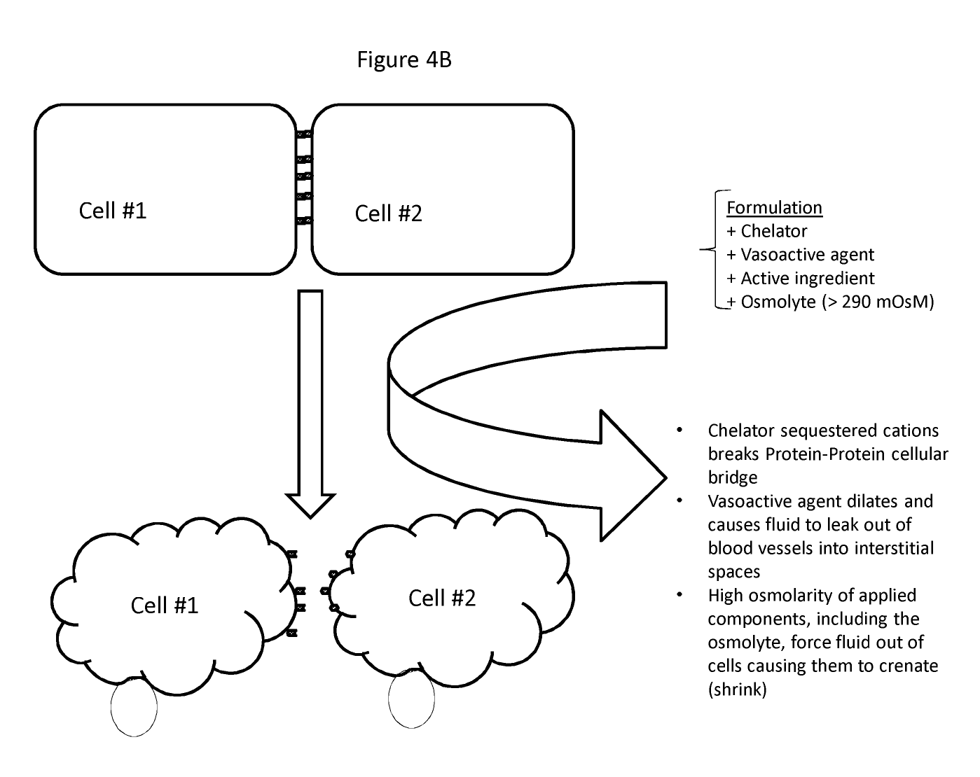

As may be readily apparent, the activities provided by the various components (e.g., in a formulation or topically applied simultaneously or sequentially) may act synergistically to transiently break the tight junctions between adjacent cells and transiently widen the interstitial spaces between the cells of the skin or of the tissue surface (without permanently damaging the cells). This allows the component with active ingredient activity in the formulation to move into the underlying tissue (e.g., the dermis or the core of a tissue such as a solid tumor). This synergy is depicted in FIG. 4. The component with chelating activity sequesters the free cations in the interstitial space, preventing the formation of new intercellular bridges and forcing apart existing bridges. Meanwhile, the high osmolarity of the combined components, whether in a formulation or applied separately (either simultaneously or sequentially) results in an increase of solutes in the interstitial space, stimulating the cells to release water into those spaces in an attempt to restore the equilibrium of solutes across the cell membrane. Consequently, the cells shrink and their cell membranes become crenated, resulting in physical stress on the bridges formed between two adjacent cells. The bridges are also physically forced apart as the distance between the cells grows when the volume of fluid in the interstitial space increases with fluid escaping from blood vessels responding to the component with vasoactive activity (e.g., a vasodilator). These physical stresses forces the bridged cell surface receptors apart thereby exposing the cations holding them together. These exposed cations can then be sequestered by the chelator. Thus, in a cycle is created of sequestered cation>strained existing bridge>broken bridge with freed cation>sequestered cation, etc. is formed. The creation of such a cycle was a surprising discovery because it was unexpected that the physical stresses on the inter-cellular bridges would cause the bridging cations to be exposed to the chelator. When that cation is sequestered and the strained bridge is broken, the surrounding intact bridges have additional stress put on them because they have to bear the strain formerly borne by the now-broken bridge. This cascade of one breaking bridge accelerating the breaking of additional bridges was unexpected. By breaking down the inter-cellular bridges that form tight junctions between adjacent cells, the components in the formulations and methods described herein allow the component with active ingredient activity to move past endothelial cells, past the basement membrane, and into the underlying tissue (e.g., the dermis or core of a solid tumor).

Note that where the components are combined into a single formulation, the combination of the component with vasodilating activity with the component with chelating activity, where the formulation has an overall osmolarity that is hypertonic to the subject, can generate a larger gradient than osmotic pressure generated from the epidermal cell water movement alone and can induce greater physical space between the epithelial cells in the epidermis in which drug molecules can move. This combined and elevated osmotic pressure can continue to drive the active ingredient through the basement membrane and into the dermis to deliver the active ingredient to dermal or subcutaneous tissues local to the topical application site, or to the lymphatic system and blood capillaries for systemic distribution throughout the body of the patient.

Finally, as shown in FIG. 4, it should be noted that after application of the components (whether in a single formulation or separately), the cells are not permanently damaged. They shrink and their cell membranes become crenated, but the intact cell is not punctured or otherwise compromised. Even the cell surface molecules that formed the inter-cellular bridges are not permanently damaged. When the components of the formulation (or methods) described herein are removed through the normal interstitial fluid flow, the volume of the interstitial spaces will reduce, the intracellular volume of the cell will increase and free cations will return. The cells will resume their original shape and size and the cell surface receptors on adjacent cells, now physically closer with the swelling cells and shrinking interstitial space volume, will employ the free cations to reconstruct the intercellular bridges.

Animal models can be used to evaluate the effectiveness of a topically applied formulation in penetrating the skin tissue or tissue surface for delivery of the active ingredient, whether that active ingredients stays local to the application site or enters the patient's body systemically via, for example, the circulatory or lymphatic systems. Animal models that are preferred include pigs, guinea pigs, rabbit and mini-pigs. An example of the procedure used for such a study using guinea pigs is as follows: Male Hartley guinea pigs (250-300 g) are shaved on the back, and an area of 4×4 cm is depilated with Nair depilatory cream. After approximately 24 hours, 0.5 g of test active ingredient (e.g., in a topical formulation) is applied to the 4×4 cm area and covered with an occlusive wrap as a transpiration barrier. At 1, 2, 4, 8 and 24 hours after application, groups of 5 or more animals are anesthetized with isoflurane, the application area is swabbed with alcohol, blood is removed by cardiac stick, and the skin tissue of the application area is excised. One group of animals is anesthetized and blood and skin tissue are removed as vehicle control. Blood samples are processed and analyzed for the presence of an active ingredient via high performance liquid chromatography (HPLC). The skin below the site of application of the active ingredient (or the formulation containing the active ingredient) on each animal group is excised, weighed, homogenized in a mixture of acetonitrile and 0.1N HCl (50:50 v/v), centrifuged, and the extract analyzed for the presence of active ingredient via HPLC. The amount of active ingredient in the blood and the amount of active ingredient in the skin tissue or tissue surface may be compared to give information about the pharmacokinetics of the active ingredient. For example, for local delivery to skin tissue (e.g., a skin tumor or lesion), a higher amount in the skin relative to the blood is more efficacious, whereas when the goal is systemic delivery of the active ingredient, a higher distribution in the blood is more efficacious.

In another embodiment, a kit for topical delivery of an active ingredient to a patient is provided. The kit's components include a vasoactive agent, a chelator, an active ingredient; and optionally one or more additional components (e.g., a transpiration barrier and/or an osmolyte), where the components are selected so that none of the other components is sequestered by the chelator and where the osmolarity of all the components of the kit is greater than about 345 milliOsmol/liter; and a set of written instructions for use, by or on said patient, of the components of the kit according to one of the methods of topical delivery described herein.

In another embodiment, a kit for topical delivery of an active ingredient to a patient includes components including a vasoactive agent, a chelator, an active ingredient, an osmolyte, and optionally one or more additional components (e.g., a transpiration barrier), where the components are selected so that none of the other components is sequestered by the chelator and , optionally one or more additional components (e.g., a transpiration barrier) whereby the osmolarity of the osmolyte in the kit is greater than about 290 milliOsmol/liter, and a set of written instructions for use, by or on the patient, of the components of the kit according to one of the methods of topical delivery described herein.

In another embodiment, there is a method of manufacturing a medicament for topical delivery of an active ingredient. The method includes combining components including a vasoactive agent, a chelator, an active ingredient, and optionally one or more additional components (e.g., a transpiration barrier or an osmolyte), where the components are selected so that none of the other components is sequestered by the chelator and, where all of the components are present in sufficient amounts to raise the osmolarity of the medicament containing the active ingredient to at least about345 mOsmol/liter.

In another embodiment, there is a method of manufacturing a medicament for topical delivery of an active ingredient. The method includes combining a vasoactive agent, a chelator, an active ingredient, an osmolyte, and optionally one or more additional components (e.g., a transpiration barrier), where the components are selected so that none of the other components is sequestered by the chelator and, where the osmolyte is present in the medicament at an osmolarity of at least 290 milliOsmol/liter.

The following are some exemplary topical formulations, procedures for preparing the formulations, and possible uses for the formulations.

### EXAMPLE 1

In this Example 1, the following components were mixed together to form a formulation. As in Example 1 above, the amounts shown are in % as weight/volume, where 1% w/v is 1 gram in 100 grams.

TABLE 3
Example 1

| Component | Amount (in Percent w/w) | |
|---|---|---|
| Pemulen TR-1(2.5% solution) | 15% | w/w |
| phospholiphone 90 H | 5% | w/w |
| Methylparaben | 0.2% | w/w |
| EDTA | 0.3% | w/w |
| Urea | 4% | w/w |
| methyl nicotinate (1% solution) | 2% | w/w |
| Arginine | 1.2% | w/w |
| Menthol | 5% | w/w |
| Eucalyptol | 5% | w/w |
| Phenoxyethanol | 0.7% | w/w |
| SD-40 | 10% | w/w |
| cremophor RH-40 | 4% | w/w |
| N-Methyl Pyrrole | 3% | w/w |
| olive oil | 3% | w/w |
| vitamin E TPGS | 2% | w/w |
| Benfotiamine | 2% | w/w |
| Propylparaben | 0.1% | w/w |
| Water | 37.5% | w/w |

| | |
|---|---|
| Total | 100% |

By topically applying the formulation described in this Example 1 to the skin of a guinea pig, no skin cells were permanently damaged. The active ingredient contained within this formulation was able to move past the skin cells into the dermis. Using this procedure, an effective amount of the active ingredient (in this case, benfotiamine) was delivered into the dermis of the patient (in this case, a guinea pig) at the topical application site (in this case, the back).

### EXAMPLE 2

In this example 2, the following components were mixed together to form a formulation. The amounts shown are in % as weight/volume, where 1% w/v is 1 gram in 100 grams.

TABLE 4
Example 2

| Component | Amount (in Percent w/w) | |
|---|---|---|
| Pemulen TR-1(2.5% solution) | 8% | w/w |
| phospholiphone 90 H | 5% | w/w |
| Methylparaben | 0.2% | w/w |
| EDTA | 0.5% | w/w |
| Urea | 12% | w/w |
| Citric acid | 0.52% | w/w |
| methyl nicotinate (1% solution) | 2% | w/w |
| Arginine | 2% | w/w |
| Soybean oil | 5% | w/w |
| Crodamol DA | 5% | w/w |
| Phenoxyethanol | 0.7% | w/w |
| cremophor RH-40 | 2% | w/w |
| Glyceryl monostearate | 5% | w/w |
| vitamin E TPGS | 2% | w/w |
| Cetyl alcohol | 2% | w/w |
| Propylparaben | 0.1% | w/w |
| Gabapentin | 5% | w/w |
| Water | 42.98% | w/w |

| | |
|---|---|
| Total | 100% |

Using the formulation described in this Example 2, no skin cells were permanently damaged with the topical application of this formulation. The active ingredients contained within this formulation is able to move past the epidermal cells into the dermis. Using this procedure, an effective amount of the active ingredient (in this case, gabapentin) was delivered into the dermis of the patient (in this case, a guinea pig) at the topical application site (in this case, the back).

### EXAMPLE 3

In this example 3, the following components were mixed together to form a formulation. The amounts shown are in % as weight/volume, where 1% w/v is 1 gram in 100 grams.

TABLE 5
Example 3

| Component | Amount (in Percent; w/w) | |
|---|---|---|
| phospholiphone 90 H | 2% | w/w |
| Methylparaben | 0.2% | w/w |
| EGTA | 4% | w/w |
| Urea | 6% | w/w |
| methyl nicotinate (1% solution) | 2% | w/w |
| arginine | 2% | w/w |
| menthol | 5% | w/w |
| eucalyptol | 5% | w/w |
| phenoxyethanol | 0.7% | w/w |
| NaOH | 0.8% | w/w |
| N-Methyl Pyrrole | 3% | w/w |
| olive oil | 4% | w/w |
| vitamin E TPGS | 2% | w/w |
| steareth-20 | 2% | w/w |
| propylparaben | 0.1% | w/w |
| Stearic acid | 8% | w/w |
| stearyl alcohol | 2% | w/w |
| resveratrol | 0.5% | w/w |
| gelcarin GP379 NF | 0.5% | w/w |
| water | 50.2% | w/w |

| | |
|---|---|
| Total | 100% |

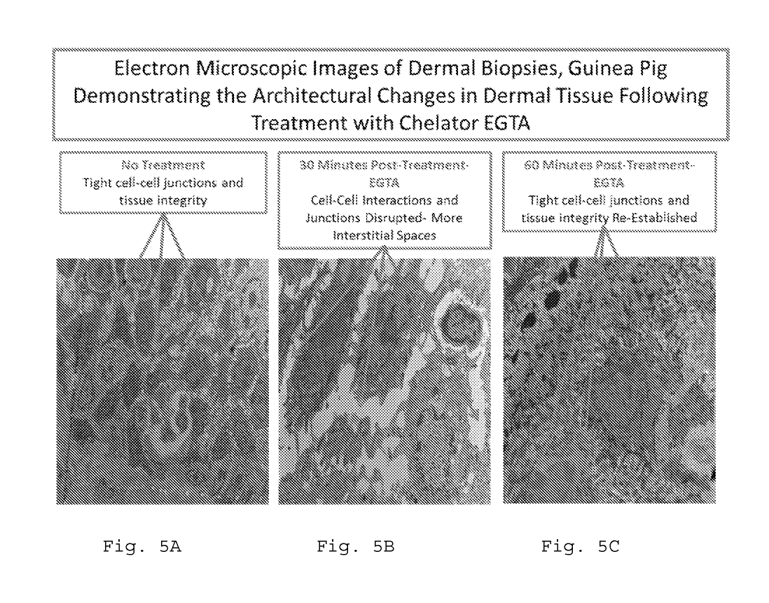

Using the guinea pig method described above, the formulation described in this Example 3 was topically applied to the back of a guinea pig after the hair was removed as described above. The results are shown in the electron microscopy images provided in FIGS. 5A, 5B, and 5C. As shown in FIG. 5A, normal skin tissue (i.e., prior to application of the formulation) shows normal tight junctions between skin cells and normal tissue structural integrity. However, thirty minutes after the topical application of the formulation, inter-cell bridges are opened and the volume of fluid in the interstitial spaces has increased (See FIG. 5A). Interestingly, sixty minutes after treatment with the formulation, the inter-cellular bridges have reformed and the tight junctions between the cells have been re-established, with the volume in the interstitial spaces reduced back to pre-treatment levels (see FIG. 5C).

Thus, using the formulation described in this Example 3, no skin cells were permanently damaged with the topical application of this formulation. The active ingredient contained within this formulation was able to move past the epidermal cells into the dermis. Using this procedure, an effective amount of the active ingredient (in this case, resveratrol) was delivered into the dermis of the patient (in this case, a guinea pig) at the topical application site (in this case, the back).

### EXAMPLE 4

In this example, the following components were mixed together to form a formulation. The amounts shown are in % as weight/volume, where 1% w/v is 1 gram in 100 grams.

TABLE 6
Example 4

| Component | Amount (in Percent; w/w) | |
|---|---|---|
| phenoxyethanol | 0.7% | w/w |
| Glycerin | 2% | w/w |
| Methylparaben | 0.2% | w/w |
| EGTA | 2% | w/w |
| Urea | 12% | w/w |
| methyl nicotinate (1% solution) | 2% | w/w |
| arginine | 2% | w/w |
| Xanthan gum | 1% | w/w |
| Gabapentin | 5% | w/w |
| Water | 73.1% | w/w |

| | |
|---|---|
| Total | 100% |

Using the formulation described in this Example 4, no skin cells were permanently damaged with the topical application of this formulation. The active ingredients contained within this formulation are able to move past the epidermal cells into the dermis. Using this procedure, an effective amount of the active ingredient (in this case, gabapentin) was delivered into the dermis of the patient (in this case, a guinea pig) at the topical application site (in this case, the back).

### EXAMPLE 5

In this example, a topical formulation comprising an active ingredient that is a biologic, namely etanercept (sold under the tradename Enbrel) is topically delivered to the knuckles and knees of human rheumatoid arthritis patients.

A formulation is made according to the formulation described in Example 3, except that the 0.5% w/w resveratrol is replaced by 0.5% w/w etanercept.

The formulation is topically applied to the skin covering the knuckles (on the patients' hands) and knees of the patients to alleviate their rheumatoid arthritis symptoms. The convenience of the topical formulation is that the patient can self-medicate (within the confines of their physician's directions) as appropriate (e.g., more frequent application on high pain days and fewer applications on days when the pain is less severe).

### EXAMPLE 6

In this example 6, a formulation containing a vasoactive agent and active ingredient, but no osmolyte, was compared to a formulation containing a vasoactive agent, an active ingredient, and an osmolyte.

Specifically, three separate topical formulations (A, B, and C) were prepared, each containing 1% ibuprofen as the active ingredient (API).

TABLE 7
Example 6

| | Formulation A | Formulation B | Formulation C |
|---|---|---|---|
| Ibuprofen (active | 1% | 1% | 1% |
| ingredient)) | | | |
| Tolazoline HCl | 0.001% | 0% | 0.001% |
| (vasodilator) | | | |
| Sorbitol (Osmolyte) | 0% | 4% | 4% |
| Veegum HV | 3% | 3% | 3% |
| Soy Bean Oil | 2% | 2% | 2% |
| Stearyl Alcohol | 5% | 5% | 5% |
| PEG-100 Stearate | 2.3% | 2.3% | 2.3% |
| Glyceryl Monostearate | 1.2% | 1.2% | 1.2% |
| Propyl Paraben | 0.1% | 0.1% | 0.1% |
| Phenoxyethanol | 0.7% | 0.7% | 0.7% |
| Methyl Paraben | 0.2% | 0.2% | 0.2% |
| KOH | 0.27% | 0.27% | 0.27% |
| Water | 84.229% | 80.23% | 80.229% |
| Total (g) | 100% | 100% | 100% |

The first topical ibuprofen formulation, Formulation A, contained 0.001% tolazoline as the vasoactive agent but no osmolyte (see Table 8 above). The second topical ibuprofen formulation (Formulation B) contained 4% w/w sorbitol (a sugar osmolyte) but no vasoactive agent. The third ibuprofen formulation (Formulation C) contained both 0.001% w/w tolazoline (vasoactive agent) and 4% w/w sorbitol (osmolyte). The total osmolarity of Formulation C was 376 milliOsmoles/Liter (mOsm/L).

Each of Formulations A, B, and C was applied to the dorsal skin of guinea pigs (n=5 per group). Two hours after the single application of the topical formulations to the animals, blood samples were collected; plasma was prepared and subsequently analyzed for the concentration of ibuprofen in the plasma (shown in Table 9 below as ugrams/ml plasma). The presence of the active ingredient (ibuprofen) in the plasma indicates that the active ingredient was able to be transported through the skin and into the underlying tissue.

The results are shown in Table 8.

TABLE 8

| | | Ibuprofen (API) | |
|---|---|---|---|
| | % w/w of API, | Plasma Concen- | |
| Formu- | vasodilator, | tration | Notes of |
| lation | and osmolyte | (ugram/ml) | Composition |
| A | 1% API & | 0.03 | Active Ingredient, Plus |
| | 0.001% | | Vasodilator Without |
| | vasodilator | | osmolyte |
| B | 1% API & 4% | 0.06 | Active Ingredient, Plus |
| | osmolyte | | Osmolyte Without |
| | | | vasodilator |
| C | 1% API & | 0.48 | Active Ingredient, Plus |
| | 0.001% | | Osmolyte, Plus |
| | vasodilator & 4% | | vasodilator |
| | osmolyte | | |

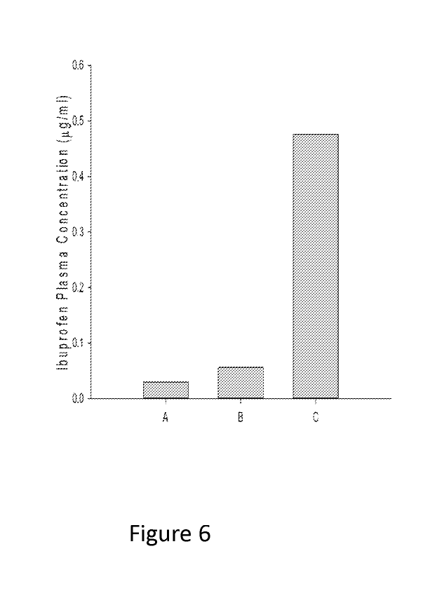

The results are depicted graphically in FIG. 6. As shown in Table 8 and FIG. 6, Formulation A containing 0.001% w/w tolazoline (vasoactive agent) and 1% w/w ibuprofen but no osmolyte produced a plasma concentration of 0.03 μg ibuprofen/ml plasma. Formulation B containing 4% w/w sorbitol (osmolyte) and 1% w/w ibuprofen but no vasoactive agent produced a plasma level of 0.06 μg ibuprofen /ml plasma. However, Formulation C. containing 1% w/w ibuprofen, 4% sorbitol (osmolyte), and 0.001% tolazoline (vasoactive agent) produced a plasma concentration of 0.48 ug ibuprofen/ml plasma.

These results in Table 8 and FIG. 6 show that the components in Formulation C are acting synergistically to drive the ibuprofen active ingredient through the skin surface, through the epidermis and dermis, and into the systemic circulation (in this case, the blood system). If the components of Formulation C were simply acting additively to facilitate transport of the active ingredient through the skin and into the underlying tissue, one would have expected Formulation C to result in 0.09 ug ibuprofen/ml plasma (i.e., 0.03 ug/ml from Formulation A plus 0.06 ug/ml from Formulation B).

### EXAMPLE 7

In this example, an anti-cancer therapeutic is topically applied to the surface of a solid tumor.

A formulation is prepared in with the present disclosure, in Examples 1-5, using as the active ingredient with one or more (in a combination therapy) of the following active ingredients that has been approved for the treatment of non-Hodgkin's lymphoma: Abitrexate Methotrexate); Adcetris (Brentuximab Vedotin); Adriamycin PFS (Doxorubicin Hydrochloride); Adriamycin RDF (Doxorubicin Hydrochloride); Ambochlorin (Chlorambucil); Amboclorin (Chlorambucil); Arranon (Nelarabine); Bendamustine Hydrochloride; Bexxar (Tositumomab and Iodine I 131 Tositumomab); Blenoxane (Bleomycin); Bleomycin; Bortezomib; Brentuximab Vedotin; Chlorambucil; Clafen (Cyclophosphamide); Cyclophosphamide; Cytoxan (Cyclophosphamide); Denileukin Diftitox; DepoCyt (Liposomal Cytarabine); Doxorubicin Hydrochloride; DTIC-Dome (Dacarbazine); Folex (Methotrexate); Folex PFS (Methotrexate); Folotyn (Pralatrexate); Ibritumomab Tiuxetan; Intron A (Recombinant Interferon Alfa-2b); Istodax (Romidepsin); Leukeran (Chlorambucil); Linfolizin (Chlorambucil); Liposomal Cytarabine; Matulane (Procarbazine Hydrochloride); Methotrexate; Methotrexate LPF (Methotrexate); Mexate (Methotrexate); Mexate-AQ (Methotrexate); Mozobil (Plerixafor); Nelarabine; Neosar (Cyclophosphamide); Ontak (Denileukin Diftitox); Plerixafor; Pralatrexate; Recombinant Interferon Alfa-2b; Rituxan (Rituximab); Rituximab; Romidepsin; Tositumomab and Iodine I 131 Tositumomab; Treanda (Bendamustine Hydrochloride); Velban (Vinblastine Sulfate); Velcade (Bortezomib); Velsar (Vinblastine Sulfate); Vinblastine Sulfate; Vincasar PFS (Vincristine Sulfate); Vincristine Sulfate; Vorinostat; Zevalin (Ibritumomab Tiuxetan); or Zolinza (Vorinostat).

Human patients with non-Hodgkin lymphoma are identified. Palpable tumors in the lymph nodes are targeted. The skin and tissue covering the lymphoma are pulled aside (e.g., in surgery with a scalpel and forceps), and the solid tumor identified. A formulation as described in herein (e.g., as in Examples 1-5) is sterilized (e.g., by passage through a filter of 0.2um) and applied to the surface of the tumor. The skin and tissue covering the lymphoma is replaced and stapled or sutured to close the wound.

The tumor is measured (e.g., with calipers) to determine if the topically applied formulation is successful in reducing the size and/or volume of the tumor.

The embodiments of the invention described above are intended to be merely exemplary; numerous variations and modifications will be apparent to those skilled in the art. All such variations and modifications are intended to be within the scope of the present invention as defined in any appended claims.

1. A topical formulation for delivery of an active ingredient to a patient, comprising components including:
a) an active ingredient;
b) a vasoactive agent; and
c) a chelator, wherein the components are selected so that none of the other components is sequestered by the chelator,
wherein the formulation comprises an osmolarity that is greater than about 345 milliOsmoles/liter (345 mOsM).

2. The formulation of claim 1, wherein the active ingredient, vasoactive agent, and chelator are mutually exclusive.

3. The formulation of claim 1, comprising an osmolyte, wherein the osmolyte does not include an ion with a valency higher than monovalency.

4. The formulation of claim 3, wherein the osmolyte is a sugar osmolyte.

5. The formulation of claim 1, wherein the active ingredient, vasoactive agent, chelator, and osmolyte are mutually exclusive.

6. The formulation of claim 1, further comprising a lipid.

7. The formulation of claim 1, further comprising a tissue penetration enhancer.

8. The formulation of claim 1, wherein the chelator is EDTA or EGTA.

9. The formulation of claim 8, wherein the concentration of the chelator is between about 0.05% w/w and about 10% w/w.

10. The formulation of claim 8, wherein the concentration of the chelator is between about 1% w/w and about 5% w/w.

11. The formulation of claim 1, wherein the vasoactive agent is a vasodilator.

12. The formulation of claim 1, wherein topical application of the formulation to the patient does not permanently damage cells of the patient.

13. A topical formulation for delivery of an active ingredient to a patient, comprising components including:
a) an active ingredient;
b) a vasoactive agent;
c) a chelator, wherein the components are selected so that none of the other components is sequestered by the chelator; and
d) an osmolyte, wherein the osmolyte does not include an ion with a valency higher than monovalency,
wherein the osmolyte comprises an osmolarity that is greater than about 290 milliOsmoles/liter (290 mOsM).

14. The method of claim 13, wherein the active ingredient, vasoactive agent, chelator, and osmolyte are mutually exclusive.

15. The formulation of claim 13, further comprising a lipid.

16. The formulation of claim 13, further comprising a tissue penetration enhancer.

17. The formulation of claim 13, wherein the chelator is EDTA or EGTA.

18. The formulation of claim 17, wherein the concentration of the chelator is between about 0.05% w/w and about 10% w/w.

19. The formulation of claim 17, wherein the concentration of the chelator is between about 1% w/w and about 5% w/w.

20. The formulation of claim 13, wherein the vasoactive agent is a vasodilator.

21. The formulation of claim 13, wherein the osmolyte is a sugar osmolyte.

22. The formulation of claim 13, wherein topical application of the formulation to the patient does not permanently damage cells of the patient.

23. A method for topical delivery of an active ingredient to a patient in need thereof, comprising applying an effective amount of the formulation of claim 1 to a topical application site of the patient.

24. A method for topical delivery of an active ingredient to a patient in need thereof, comprising
a) applying an effective amount of the active ingredient to a topical application site of the patient;
b) applying a first amount of a vasoactive agent to the topical application site; and
c) applying a second amount of a chelator to the topical application site, wherein the vasoactive agent and the active ingredient are selected so none of the vasoactive agent and the active ingredient is sequestered by the chelator;
wherein the first amount and second amount, together with the effective amount, result in an osmolarity of the components at the topical application site of at least 345 mOsmol/liter.

25. The method of claim 24, further comprising applying a third amount of an osmolyte to the topical application site, wherein the osmolyte does not include an ion with a valency higher than monovalency.

26. The method of claim 25, wherein the osmolyte is present at an osmolarity of at least 290 mOsmol/liter.

27. The method of claim 25, wherein the osmolyte is a sugar osmolyte.

28. The method of claim 24, wherein the active ingredient, vasoactive agent, and chelator are applied sequentially.

29. The method of claim 24, wherein the active ingredient, vasoactive agent, and chelator are applied together.

30. The method of claim 23, wherein cells at the topical application site are not permanently damaged.

31. The method of claim 23, wherein the patient is human.

32. The method of claim 23, wherein the topical application site is on a skin surface of the patient.

33. The method of claim 23, wherein the topical application site is on a tissue surface of a patient.

34. The method of claim 33, wherein the tissue surface is a surface of a solid tumor.

35. The method of claim 33, wherein the tissue surface is a surface of an organ.

36. A kit for topical delivery of an active ingredient to a patient, comprising components having a combined osmolarity that is greater than 345 mOsmole/liter, the components including
a) the active ingredient;
b) a vasoactive agent; and
c) a chelator, wherein the components are selected so that none of the other components is sequestered by the chelator; and
d) a set of written instructions for use, by or on said patient, of the components of the kit.

37. A kit for topical delivery of an active ingredient to a patient, comprising components having a combined osmolarity that is greater than 345 mOsmole/liter, the components including
a) the active ingredient;
b) a vasoactive agent;
c) an osmolyte in an amount where the osmolarity of the osmolyte is at least 290 mOsmol/liter; and
d) a chelator, wherein the components are selected so that none of the other components is sequestered by the chelator; and
e) a set of written instructions for use, by or on said patient, of the components of the kit.

38. A method of manufacturing a medicament for topical delivery of an active ingredient, comprising combining components including:
a) the active ingredient;
b) a vasoactive agent; and
c) a chelator, wherein the components are selected so that none of the other components is sequestered by the chelator;
where all of the components are present in sufficient amounts to raise the osmolarity of the medicament containing the active ingredient to at least about 345 mOsmol/liter.

39. A method of manufacturing a medicament for topical delivery of an active ingredient, comprising combining components including:
a) the active ingredient;
b) a vasoactive agent; and
c) a chelator, wherein the components are selected so that none of the other components is sequestered by the chelator; and
an osmolyte, where the osmolyte is present in the medicament at an osmolarity of at least 290 milliOsmol/liter.

40. The method of claim 24, wherein cells at the topical application site are not permanently damaged.

41. The method of claim 24, wherein the patient is human.

42. The method of claim 24, wherein the topical application site is on a skin surface of the patient.

43. The method of claim 24, wherein the topical application site is on a tissue surface of a patient.
